SEQUENCE LISTING (24 sequences)

SEQ ID NO: 1 - protein, 10 aa, synthetic construct
  REGION 1..10: note = Description of Artificial Sequence: Synthetic peptide
GYTFTNYDIH

SEQ ID NO: 2 - protein, 17 aa, synthetic construct
  REGION 1..17: note = Description of Artificial Sequence: Synthetic peptide
WIYPGDGSTKYNEKFKG

SEQ ID NO: 3 - protein, 4 aa, synthetic construct
  REGION 1..4: note = Description of Artificial Sequence: Synthetic peptide
EWAY

SEQ ID NO: 4 - protein, 11 aa, synthetic construct
  REGION 1..11: note = Description of Artificial Sequence: Synthetic peptide
RASQDIGGNLY

SEQ ID NO: 5 - protein, 7 aa, synthetic construct
  REGION 1..7: note = Description of Artificial Sequence: Synthetic peptide
ATSSLDS

SEQ ID NO: 6 - protein, 9 aa, synthetic construct
  REGION 1..9: note = Description of Artificial Sequence: Synthetic peptide
LQYSSSPWT

SEQ ID NO: 7 - protein, 462 aa, synthetic construct
  REGION 1..462: note = Description of Artificial Sequence: Synthetic polypeptide
MDWTWRVFCLLAVAPGAHSQVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 234 aa, synthetic construct
  REGION 1..234: note = Description of Artificial Sequence: Synthetic polypeptide
METPAQLLFLLLLWLPDTTGDIQMTQSPSSLSASLGERVSLTCRASQDIGGNLYWLQQGPDGTIKRLIYATSSLDSGVPKRFSGSRSGSDYSLTISSLESEDFVDYYCLQYSSSPWTFGGGTKMEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 654 aa, synthetic construct
  REGION 1..654: note = Description of Artificial Sequence: Synthetic polypeptide
LRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 10 - protein, 1119 aa, synthetic construct
  REGION 1..1119: note = Description of Artificial Sequence: Synthetic polypeptide
MDWTWRVFCLLAVAPGAHSQVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSSSSLRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 11 - protein, 1146 aa, synthetic construct
  REGION 1..1146: note = Description of Artificial Sequence: Synthetic polypeptide
MDWTWRVFCLLAVAPGAHSQVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSSSELKTPLGDTTHTSPRSPAPEFLGGPSSSLRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 12 - DNA, 1965 bp, synthetic construct
  misc_feature 1..1965: note = Description of Artificial Sequence: Synthetic polynucleotide
  source 1..1965: mol_type = other DNA
ttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatga

SEQ ID NO: 13 - DNA, 1398 bp, synthetic construct
  misc_feature 1..1398: note = Description of Artificial Sequence: Synthetic polynucleotide
  source 1..1398: mol_type = other DNA
gccgccaccatggactggacctggagggtgttctgcctgcttgcagtggcccccggagcccacagccaggttcagctgcagcagtctggacctgagctggtgaagcctggggctttagtgaagatatcctgcaaggcttctggttacaccttcacaaactacgatatacactgggtgaagcagaggcctggacagggacttgagtggattggatggatttatcctggagatggtagtactaagtacaatgagaaattcaagggcaaggccacactgactgcagacaaatcctccagcacagcctacatgcacctcagcagcctgacttctgagaaatctgcagtctatttctgtgcaagagagtgggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtaagtaa

SEQ ID NO: 14 - DNA, 714 bp, synthetic construct
  misc_feature 1..714: note = Description of Artificial Sequence: Synthetic polynucleotide
  source 1..714: mol_type = other DNA
gccgccaccatggagacccccgcccagctgctgttcctgttgctgctttggcttccagatactaccggcgacatccagatgacccagtctccatcctccttatctgcctctctgggagaaagagtcagtctcacttgtcgggcaagtcaggacattggtggtaacttatactggcttcagcagggaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacagtattctagttctccgtggacgttcggtggaggcacaaagatggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 15 - DNA, 3450 bp, synthetic construct
  misc_feature 1..3450: note = Description of Artificial Sequence: Synthetic polynucleotide
  source 1..3450: mol_type = other DNA
gccgccaccatggactggacctggagggtgttctgcctgcttgcagtggcccccggagcccacagccaggttcagctgcagcagtctggacctgagctggtgaagcctggggctttagtgaagatatcctgcaaggcttctggttacaccttcacaaactacgatatacactgggtgaagcagaggcctggacagggacttgagtggattggatggatttatcctggagatggtagtactaagtacaatgagaaattcaagggcaaggccacactgactgcagacaaatcctccagcacagcctacatgcacctcagcagcctgacttctgagaaatctgcagtctatttctgtgcaagagagtgggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtagtagttcagagctcaaaaccccacttggtgacacaactcacacaagcccacggagcccagcacctgaattcctggggggaccgagttcctcattgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatga

SEQ ID NO: 16 - DNA, 573 bp, synthetic construct
  misc_feature 1..573: note = Description of Artificial Sequence: Synthetic polynucleotide
  source 1..573: mol_type = other DNA
gccgccaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaa

SEQ ID NO: 17 - protein, 187 aa, synthetic construct
  REGION 1..187: note = Description of Artificial Sequence: Synthetic polypeptide
MVRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 18 - protein, 4 aa, synthetic construct
  REGION 1..4: note = Description of Artificial Sequence: Synthetic peptide
SSSS

SEQ ID NO: 19 - protein, 31 aa, synthetic construct
  REGION 1..31: note = Description of Artificial Sequence: Synthetic polypeptide
SSSELKTPLGDTTHTSPRSPAPEFLGGPSSS

SEQ ID NO: 20 - protein, 15 aa, synthetic construct
  REGION 1..15: note = Description of Artificial Sequence: Synthetic peptide
GGGGSGGGGSGGGGS

SEQ ID NO: 21 - protein, 23 aa, synthetic construct
  REGION 1..23: note = Description of Artificial Sequence: Synthetic peptide
SSSELKTPLGDTTHTSPRSPSSS

SEQ ID NO: 22 - protein, 58 aa, synthetic construct
  REGION 1..58: note = Description of Artificial Sequence: Synthetic polypeptide
SSSELKTPLGDTTHTSPRSPAPEFLGGPSSELKTPLGDTTHTSPRSPAPEFLGGPSSS

SEQ ID NO: 23 - protein, 56 aa, synthetic construct
  REGION 1..56: note = Description of Artificial Sequence: Synthetic polypeptide
SELKTPLGDTTHTSPRSPAPEFLGGPSSELKTPLGDTTHTSPRSPAPEFLGGPSSS

SEQ ID NO: 24 - protein, 5 aa, synthetic construct
  REGION 1..5: note = Description of Artificial Sequence: Synthetic peptide
GGGGS

(19) United States
(12) Patent Application Publication
(10) Pub. No.: US 2025/0179201 A1
(43) Pub. Date: Jun. 5, 2025

(54) METHODS AND COMPOSITIONS FOR INCREASING GALACTOSIDASE BETA-1 ACTIVITY IN THE CNS

(71) Applicant: ArmaGen, Inc., San Diego, CA (US)

(72) Inventors: William M. PARDRIDGE, Pacific Palisades, CA (US); Ruben J. BOADO, Agoura Hills, CA (US)

(73) Assignee: ArmaGen, Inc., San Diego, CA (US)

(21) Appl. No.: 19/039,422

(22) Filed: Jan. 28, 2025

Related U.S. Application Data
(63) Continuation of application No. 17/295,390, filed on May 19, 2021.
(60) Provisional application No. 62/789,953, filed on Jan. 8, 2019.

(51) Int. Cl. C07K 16/28; A61K 38/47; A61K 39/00; A61P 25/00; C12N 9/38

(52) U.S. Cl. CPC C07K 16/2869; A61K 38/47; A61P 25/00; C12N 9/2471; C12Y 302/01023; A61K 2039/505; A61K 2039/545; C07K 2317/565; C07K 2317/92; C07K 2319/30; C07K 2319/75

(57) ABSTRACT

Provided herein are methods and compositions for treating a subject suffering from an enzyme deficiency in the central nervous system (CNS). The bifunctional fusion antibody provided herein comprise an antibody to an endogenous blood brain barrier (BBB) receptor and an enzyme deficient in GM1 gangliosidosis or GM1. The fusion antibodies provided herein comprise galactosidase beta-1 (GLB1). The methods of treating an enzyme deficiency in the CNS comprise systemic administration of a fusion antibody provided herein.

### CROSS-REFERENCE TO RELATED APPLICATIONS

This application is a continuation of Ser. No. 17/295,390, filed May 19, 2021, which is a U.S. National Stage Entry of PCT/US2020/012549, filed Jan. 7, 2020, which claims the benefit of U.S. Provisional Patent Application No. 62/789,953 filed on Jan. 8, 2019. Priority is claimed pursuant to 35 U.S.C. § 119. The above noted patent application is incorporated by reference as if set forth fully herein.

### SEQUENCE LISTING

The instant application contains a Sequence Listing which has been submitted electronically in XML file format and is hereby incorporated by reference in its entirety. Said XML copy, created on Jan. 28, 2025, is named 28570-720.301_SL.xml and is 43,847 bytes in size.

### BACKGROUND OF THE INVENTION

GM1 gangliosidosis (GM1) is an inherited metabolic disease that mainly affects the central nervous system (CNS), as well as somatic organs. GM1 disease is caused by mutations in the GLB1 gene which encodes the lysosomal enzyme, galactosidase beta-1, or GLB1. GLB1 breaks down certain glycolipids such as GM1 ganglioside, as well as certain glycosaminoglycans (GAG) such as keratin sulfate (KS). GM1 can present in infancy, in the juvenile period, and have a delayed onset, depending on the severity of inherited loss of GLB1 enzyme activity. With infantile presentation, generally by the age of 6 months, there is mental retardation, seizures, blindness, as well as somatic problems including hepatosplenomegaly, bone involvement, and cardiomyopathy, with death in early childhood. The juvenile form of the disease may present between 18 months and 5 years, with similar severity of symptoms and death follows by mid to late childhood. Typically, treatment of a lysosomal storage disorder such as GM1 would include intravenous enzyme replacement therapy, or ERT, which generally involves introduction of recombinant enzymes to replace or stand in for the patient's deficient enzymes. However, systemically administered recombinant enzymes such as GLB1 do not cross the blood brain barrier (BBB), and therefore would have little impact on the devastating effects of GM1 the CNS.

### SUMMARY OF THE INVENTION

Described herein are methods and compositions for treating a subject suffering from a deficiency of galactosidase beta-1 (“GLB1”). In certain embodiments, the methods provided herein comprise delivery of GLB1 to the CNS by systemically administering a therapeutically effective amount of a bifunctional fusion antibody or protein. In certain embodiments, the bifunctional fusion antibody comprises the amino acid sequences of an antibody to an endogenous blood brain barrier (BBB) receptor and GLB1. In some embodiments, the bifunctional fusion antibody is a human insulin receptor antibody (HIR Ab) genetically fused to GLB1 (“HIR Ab-GLB1 fusion antibody”). In certain embodiments, the HIR Ab-GLB1 fusion antibody binds to the extracellular domain of the insulin receptor and is transported across the blood brain barrier (“BBB”) into the CNS, while retaining GLB1 enzyme activity. In certain embodiments, the HIR Ab binds to the endogenous insulin receptor on the BBB, and acts as a molecular Trojan horse to ferry the GLB1 into the brain. In certain embodiments, therapeutically effective systemic dose of a HIR Ab-GLB1 fusion antibody for systemic administration is based, in part, on the specific CNS uptake characteristics of the fusion antibody from peripheral blood as described herein.

In one aspect provided herein is a method for treating an GLB1 deficiency in the central nervous system of a subject in need thereof, comprising systemically administering to the subject a therapeutically effective dose of a fusion antibody having GLB1 activity. In some embodiments, the fusion antibody comprises the amino acid sequence of an immunoglobulin heavy chain, the amino acid sequence of a GLB1, and the amino acid sequence of an immunoglobulin light chain. In some embodiments, the fusion antibody binds to an extracellular domain of an endogenous BBB receptor (e.g., the human insulin receptor) and catalyzes degradation of GM1 gangliosides. In some embodiments, the amino acid sequence of the GLB1 is covalently linked to the carboxy terminus of the amino acid sequence of the immunoglobulin heavy chain. In some embodiments, the GLB1 comprises the amino acid sequence of SEQ ID NO:9.

In some embodiments, the GLB1 retains at least 20% of its activity compared to its activity as a separate entity. In some embodiments, the GLB1 and the immunoglobulin each retains at least 20% of its activity compared to its activity as a separate entity.

In some embodiments, at least about 600 ug of GLB1 enzyme are delivered to the brain. In some embodiments at least about 900 ug of GLB1 enzyme are delivered to the brain. In some embodiments at least about 1200 ug of GLB1 enzyme are delivered to the brain. In some embodiments at least about 2000 ug of GLB1 enzyme are delivered to the brain. In some embodiments at least about 3000 ug of GLB1 enzyme are delivered to the brain. In some embodiments at least about 4000 ug of GLB1 enzyme are delivered to the brain. In some embodiments at least about 5000 ug of GLB1 enzyme are delivered to the brain. In some embodiments at least about 8000 ug of GLB1 enzyme are delivered to the brain. In some embodiments at least about 10,000 ug of GLB1 enzyme are delivered to the brain. In some embodiments at least about 300 ug of GLB1 enzyme are delivered to the brain. In some embodiments at least about 100 ug of GLB1 enzyme are delivered to the brain. In some embodiments at least about 30 ug of GLB1 enzyme are delivered to the brain. In some embodiments at least about 10 ug of GLB1 enzyme are delivered to the brain. In some embodiments at least about 3 ug of GLB1 enzyme are delivered to the brain. In some embodiments at least about 1 ug of GLB1 enzyme are delivered to the brain.

In some embodiments, at least about 1500 ug of GLB1 enzyme are delivered to the brain, normalized per 50 kg body weight. In some embodiments, at least about 2250 ug of GLB1 enzyme are delivered to the brain, normalized per 50 kg body weight. In some embodiments, at least about 3000 ug of GLB1 enzyme are delivered to the brain, normalized per 50 kg body weight. In some embodiments, at least about 5000 ug of GLB1 enzyme are delivered to the brain, normalized per 50 kg body weight. In some embodiments, at least about 7500 ug of GLB1 enzyme are delivered to the brain, normalized per 50 kg body weight. In some embodiments, at least about 10,000 ug of GLB1 enzyme are delivered to the brain, normalized per 50 kg body weight. In some embodiments, at least about 15,000 ug of GLB1 enzyme are delivered to the brain, normalized per 50 kg body weight. In some embodiments, at least about 20,000 ug of GLB1 enzyme are delivered to the brain, normalized per 50 kg body weight. In some embodiments, at least about 25,000 ug of GLB1 enzyme are delivered to the brain, normalized per 50 kg body weight. In some embodiments, at least about 750 ug of GLB1 enzyme are delivered to the brain, normalized per 50 kg body weight. In some embodiments, at least about 250 ug of GLB1 enzyme are delivered to the brain, normalized per 50 kg body weight. In some embodiments, at least about 75 ug of GLB1 enzyme are delivered to the brain, normalized per 50 kg body weight. In some embodiments, at least about 25 ug of GLB1 enzyme are delivered to the brain, normalized per 50 kg body weight. In some embodiments, at least about 7.5 ug of GLB1 enzyme are delivered to the brain, normalized per 50 kg body weight. In some embodiments, at least about 2.5 ug of GLB1 enzyme are delivered to the brain, normalized per 50 kg body weight.

In some embodiments, the therapeutically effective dose of the fusion antibody comprises at least about 0.5 mg/Kg of body weight. In some embodiments, the therapeutically effective dose of the fusion antibody comprises at least about 0.6 mg/Kg of body weight. In some embodiments, the therapeutically effective dose of the fusion antibody comprises at least about 0.7 mg/Kg of body weight. In some embodiments, the therapeutically effective dose of the fusion antibody comprises at least about 0.8 mg/Kg of body weight. In some embodiments, the therapeutically effective dose of the fusion antibody comprises at least about 0.9 mg/Kg of body weight. In some embodiments, the therapeutically effective dose of the fusion antibody comprises at least about 1 mg/Kg of body weight. In some embodiments, the therapeutically effective dose of the fusion antibody comprises at least about 3 mg/Kg of body weight. In some embodiments, the therapeutically effective dose of the fusion antibody comprises at least about 6 mg/Kg of body weight. In some embodiments, the therapeutically effective dose of the fusion antibody comprises at least about 10 mg/Kg of body weight. In some embodiments, the therapeutically effective dose of the fusion antibody comprises at least about 50 mg/Kg of body weight. In some embodiments, the therapeutically effective dose of the fusion antibody comprises at least about 0.4 mg/Kg of body weight. In some embodiments, the therapeutically effective dose of the fusion antibody comprises at least about 0.3 mg/Kg of body weight. In some embodiments, the therapeutically effective dose of the fusion antibody comprises at least about 0.2 mg/Kg of body weight. In some embodiments, the therapeutically effective dose of the fusion antibody comprises at least about 0.1 mg/Kg of body weight. In some embodiments, the therapeutically effective dose of the fusion antibody comprises at most about 100 mg/Kg of body weight. In some embodiments, the therapeutically effective dose of the fusion antibody comprises at most about 90 mg/Kg of body weight. In some embodiments, the therapeutically effective dose of the fusion antibody comprises at most about 85 mg/Kg of body weight. In some embodiments, the therapeutically effective dose of the fusion antibody comprises at most about 80 mg/Kg of body weight. In some embodiments, the therapeutically effective dose of the fusion antibody comprises at most about 75 mg/Kg of body weight. In some embodiments, the therapeutically effective dose of the fusion antibody comprises at most about 70 mg/Kg of body weight. In some embodiments, the therapeutically effective dose of the fusion antibody comprises at most about 65 mg/Kg of body weight. In some embodiments, the therapeutically effective dose of the fusion antibody comprises at most about 60 mg/Kg of body weight. In some embodiments, the therapeutically effective dose of the fusion antibody comprises at most about 50 mg/Kg of body weight. In some embodiments, the therapeutically effective dose of the fusion antibody comprises at most about 45 mg/Kg of body weight. In some embodiments, the therapeutically effective dose of the fusion antibody comprises at most about 40 mg/Kg of body weight. In some embodiments, the therapeutically effective dose of the fusion antibody comprises at most about 35 mg/Kg of body weight. In some embodiments, the therapeutically effective dose of the fusion antibody comprises at most about 30 mg/Kg of body weight. In some embodiments, the therapeutically effective dose of the fusion antibody comprises at most about 25 mg/Kg of body weight. In some embodiments, the therapeutically effective dose of the fusion antibody comprises at most about 20 mg/Kg of body weight.

In some embodiments, the therapeutically effective dose of the fusion antibody comprises at least about 100,000 units/Kg of body weight, where 1 unit of enzyme activity results in formation of 1 nmol of 4-methylumbelliferyl β-D-galactopyranoside (MUGP) per hour in the fluorometric enzyme assay (FIG. 14). In some embodiments, the therapeutically effective dose of the fusion antibody comprises at least about 120,000 units/Kg of body weight. In some embodiments, the therapeutically effective dose of the fusion antibody comprises at least about 140,000 units/Kg of body weight. In some embodiments, the therapeutically effective dose of the fusion antibody comprises at least about 160,000 units/Kg of body weight. In some embodiments, the therapeutically effective dose of the fusion antibody comprises at least about 180,000 units/Kg of body weight. In some embodiments, the therapeutically effective dose of the fusion antibody comprises at least about 200,000 units/Kg of body weight. In some embodiments, the therapeutically effective dose of the fusion antibody comprises at least about 600,000 units/Kg of body weight. In some embodiments, the therapeutically effective dose of the fusion antibody comprises at least about 1,200,000 units/Kg of body weight. In some embodiments, the therapeutically effective dose of the fusion antibody comprises at least about 2,000,000 units/Kg of body weight. In some embodiments, the therapeutically effective dose of the fusion antibody comprises at least about 10,000,000 units/Kg of body weight. In some embodiments, the therapeutically effective dose of the fusion antibody comprises at least about 80,000 units/Kg of body weight. In some embodiments, the therapeutically effective dose of the fusion antibody comprises at least about 60,000 units/Kg of body weight. In some embodiments, the therapeutically effective dose of the fusion antibody comprises at least about 40,000 units/Kg of body weight. In some embodiments, the therapeutically effective dose of the fusion antibody comprises at least about 20,000 units/Kg of body weight. In some embodiments, the therapeutically effective dose of the fusion antibody comprises at most about 20,000,000 units/Kg of body weight. In some embodiments, the therapeutically effective dose of the fusion antibody comprises at most about 19,000,000 units/Kg of body weight. In some embodiments, the therapeutically effective dose of the fusion antibody comprises at most about 18,000,000 units/Kg of body weight. In some embodiments, the therapeutically effective dose of the fusion antibody comprises at most about 17,000,000 units/Kg of body weight. In some embodiments, the therapeutically effective dose of the fusion antibody comprises at most about 16,000,000 units/Kg of body weight. In some embodiments, the therapeutically effective dose of the fusion antibody comprises at most about 15,000,000 units/Kg of body weight. In some embodiments, the therapeutically effective dose of the fusion antibody comprises at most about 14,000,000 units/Kg of body weight. In some embodiments, the therapeutically effective dose of the fusion antibody comprises at most about 13,000,000 units/Kg of body weight. In some embodiments, the therapeutically effective dose of the fusion antibody comprises at most about 12,000,000 units/Kg of body weight. In some embodiments, the therapeutically effective dose of the fusion antibody comprises at most about 11,000,000 units/Kg of body weight. In some embodiments, the therapeutically effective dose of the fusion antibody comprises at most about 10,900,000 units/Kg of body weight. In some embodiments, the therapeutically effective dose of the fusion antibody comprises at most about 10,800,000 units/Kg of body weight. In some embodiments, the therapeutically effective dose of the fusion antibody comprises at most about 10,700,000 units/Kg of body weight. In some embodiments, the therapeutically effective dose of the fusion antibody comprises at most about 10,600,000 units/Kg of body weight. In some embodiments, the therapeutically effective dose of the fusion antibody comprises at most about 10,500,000 units/Kg of body weight.

In some embodiments, the GLB1 specific activity of the fusion antibody is at least 20,000 units/mg protein. In some embodiments, the GLB1 specific activity of the fusion antibody is at least 60,000 units/mg. In some embodiments, the GLB1 specific activity of the fusion antibody is at least 120,000 units/mg. In some embodiments, the GLB1 specific activity of the fusion antibody is at least 200,000 units/mg. In some embodiments, the GLB1 specific activity of the fusion antibody is at least 500,000 units/mg. In some embodiments, the GLB1 specific activity of the fusion antibody is at least 1,000,000 units/mg. In some embodiments, the GLB1 specific activity of the fusion antibody is at least 5,000,000 units/mg.

In some embodiments, systemic administration is parenteral, intravenous, subcutaneous, intra-muscular, trans-nasal, intra-arterial, transdermal, or respiratory.

In some embodiments, the fusion antibody is a chimeric antibody. In some embodiments, the fusion antibody is a humanized antibody.

In some embodiments, the immunoglobulin heavy chain is an immunoglobulin heavy chain of IgG. In some embodiments, the immunoglobulin heavy chain is an immunoglobulin heavy chain of IgG1.

In some embodiments, the immunoglobulin heavy chain of the fusion antibody comprises a CDR1 corresponding to the amino acid sequence of SEQ ID NO:1 with up to 4 single amino acid mutations, a CDR2 corresponding to the amino acid sequence of SEQ ID NO:2 with up to 6 single amino acid mutations, or a CDR3 corresponding to the amino acid sequence of SEQ ID NO:3 with up to 3 single amino acid mutations, wherein the single amino acid mutations are substitutions, deletions, or insertions.

In other embodiments, the immunoglobulin heavy chain of the fusion antibody comprises a CDR1 corresponding to the amino acid sequence of SEQ ID NO:1 with a single amino acid mutations, a CDR2 corresponding to the amino acid sequence of SEQ ID NO:2 with a single amino acid mutations, and a CDR3 corresponding to the amino acid sequence of SEQ ID NO:3 with a single amino acid mutation.

In other embodiments, the immunoglobulin heavy chain of the fusion antibody comprises a CDR1 corresponding to the amino acid sequence of SEQ ID NO:1, a CDR2 corresponding to the amino acid sequence of SEQ ID NO:2, or a CDR3 corresponding to the amino acid sequence of SEQ ID NO:3.

In some embodiments, the immunoglobulin light chain is an immunoglobulin light chain of kappa or lambda class.

In some embodiments, the immunoglobulin light chain of the fusion antibody comprises a CDR1 corresponding to the amino acid sequence of SEQ ID NO:4 with up to 3 single amino acid mutations, a CDR2 corresponding to the amino acid sequence of SEQ ID NO:5 with up to 5 single amino acid mutations, or a CDR3 corresponding to the amino acid sequence of SEQ ID NO:6 with up to 5 single amino acid mutations, wherein the single amino acid mutations are substitutions, deletions, or insertions.

In other embodiments, the immunoglobulin light chain of the fusion antibody comprises a CDR1 corresponding to the amino acid sequence of SEQ ID NO:4 with a single amino acid mutations, a CDR2 corresponding to the amino acid sequence of SEQ ID NO:5 with a single amino acid mutations, and a CDR3 corresponding to the amino acid sequence of SEQ ID NO:6 with a single amino acid mutations.

In other embodiments, the immunoglobulin light chain of the fusion antibody comprises a CDR1 corresponding to the amino acid sequence of SEQ ID NO:4, a CDR2 corresponding to the amino acid sequence of SEQ ID NO:5, or a CDR3 corresponding to the amino acid sequence of SEQ ID NO:6.

In some embodiments, the immunoglobulin heavy chain of the fusion antibody comprises a CDR1 corresponding to the amino acid sequence of SEQ ID NO:1, a CDR2 corresponding to the amino acid sequence of SEQ ID NO:2, and a CDR3 corresponding to the amino acid sequence of SEQ ID NO:3; and the immunoglobulin light chain comprises a CDR1 corresponding to the amino acid sequence of SEQ ID NO:4, a CDR2 corresponding to the amino acid sequence of SEQ ID NO:5, and a CDR3 corresponding to the amino acid sequence of SEQ ID NO:6.

In some embodiments, the immunoglobulin heavy chain of the fusion antibody is at least 90% identical to SEQ ID NO:7 and the amino acid sequence of the light chain immunoglobulin is at least 90°/identical to SEQ ID NO:8.

In some embodiments, the immunoglobulin heavy chain of the fusion antibody is at least 95% identical to SEQ ID NO:7 and the amino acid sequence of the light chain immunoglobulin is at least 95% identical to SEQ ID NO:8.

In some embodiments, the immunoglobulin heavy chain of the fusion antibody comprises SEQ ID NO:7 and the amino acid sequence of the light chain immunoglobulin comprises SEQ ID NO:8.

In some embodiments, the GLB1 comprises an amino acid sequence at least 90% identical to SEQ ID NO:9. In some embodiments, the GLB1 comprises an amino acid sequence at least 95% identical to SEQ ID NO:9. In some embodiments, the GLB1 comprises an amino acid sequence of SEQ ID NO:9.

In other embodiments, the amino acid sequence of the immunoglobulin heavy chain of the fusion antibody at least 90% identical to SEQ ID NO:7; the amino acid sequence of the light chain immunoglobulin is at least 90% identical to SEQ ID NO:8; and the amino acid sequence of the GLB1 is at least 95% identical to SEQ ID NO:9 or comprises SEQ ID NO:9.

In other embodiments, the amino acid sequence of the immunoglobulin heavy chain of the fusion antibody comprises SEQ ID NO:7, the amino acid sequence of the immunoglobulin light chain comprises SEQ ID NO:8, and the amino acid sequence of the GLB1 comprises SEQ ID NO:9

In some embodiments, the fusion antibody provided herein crosses the BBB by binding an endogenous BBB receptor-mediated transport system. In some embodiments, the fusion antibody crosses the BBB via an endogenous BBB receptor selected from the group consisting of the insulin receptor, transferrin receptor, leptin receptor, lipoprotein receptor, and the insulin-like growth factor (IGF) receptor. In some embodiments, the fusion antibody crosses the BBB by binding an insulin receptor.

In some embodiments, the GLB1 deficiency in the central nervous system is GM1.

In some aspects, provided herein is a method for treating an GLB1 deficiency in the central nervous system of a subject in need thereof, comprising systemically administering to the subject a therapeutically effective dose of a fusion antibody having GLB1 activity, wherein the fusion antibody comprises: (a) a fusion protein comprising the amino acid sequences of an immunoglobulin heavy chain and a GLB1, and (b) an immunoglobulin light chain; wherein the fusion antibody crosses the blood brain barrier (BBB). In some embodiments, the amino acid sequence of the GLB1 is covalently linked to the carboxy terminus of the amino acid sequence of the immunoglobulin heavy chain.

In some aspects, provided herein is a method for treating an GLB1 deficiency in the central nervous system of a subject in need thereof, comprising systemically administering to the subject a therapeutically effective dose of a fusion antibody having GLB1 activity, wherein the fusion antibody comprises: (a) a fusion protein comprising an amino acid sequence that is at least 90% identical to SEQ ID NO:10, and (b) an immunoglobulin light chain. In some embodiments, the fusion antibody binds to an extracellular domain of an endogenous BBB receptor. In some embodiments, the endogenous BBB receptor is the human insulin receptor. In some embodiments, the fusion antibody catalyzes degradation of GM1 gangliosides. In some embodiments, the fusion protein comprises an amino acid sequence that is at least 95% identical to SEQ ID NO: 10. In some embodiments, the fusion protein comprises the amino acid sequence of SEQ ID NO: 10.

In some aspects, provided herein is a fusion antibody having GLB1 activity, the fusion antibody comprising (a) a fusion protein comprising an amino acid sequence that is at least 90% identical to SEQ ID NO:10, and (b) an immunoglobulin light chain. In some embodiments, the fusion antibody binds to an extracellular domain of an endogenous BBB receptor. In some embodiments, the endogenous BBB receptor is the human insulin receptor. In some embodiments, the fusion antibody is an antibody that binds to the endogenous BBB receptor. In some embodiments, the fusion antibody is an antibody that binds to the human insulin receptor. In some embodiments, the fusion antibody catalyzes degradation of GM1 gangliosides. In some embodiments, the fusion protein comprises an amino acid sequence that is at least 95% identical to SEQ ID NO: 10. In some embodiments, the fusion protein comprises the amino acid sequence of SEQ ID NO: 10.

In some aspects, provided herein is a fusion antibody having GLB1 activity, the fusion antibody comprising (a) a fusion protein comprising the amino acid sequence of an immunoglobulin heavy chain and GLB1, and (b) an immunoglobulin light chain. In some embodiments, the amino acid sequence of the GLB1 is covalently linked to either the amino terminus or the carboxy terminus of the amino acid sequence of the immunoglobulin heavy chain. In some embodiments, provided herein is a fusion antibody having GLB1 activity, the fusion antibody comprising (a) a fusion protein comprising the amino acid sequence of an immunoglobulin light chain and GLB1, and (b) an immunoglobulin heavy chain. In some embodiments, the amino acid sequence of the GLB1 is covalently linked to either the amino terminus or the carboxy terminus of the amino acid sequence of the immunoglobulin light chain. In some embodiments, the fusion antibody binds to the extracellular domain of an endogenous BBB receptor. In some embodiments, the endogenous BBB receptor is the human insulin receptor. In some embodiments, the fusion antibody is an antibody that binds to the endogenous BBB receptor. In some embodiments, the fusion antibody is an antibody that binds to the human insulin receptor. In some embodiments, the fusion antibody catalyzes degradation of GM1 gangliosides.

In some embodiments, the fusion protein provided herein further comprises a linker between the amino acid sequence of the GLB1 and the carboxy terminus of the amino acid sequence of the immunoglobulin light chain. In some embodiments, the linker is 80%, 85%, 90%, 95%, 96%, 97%, 98%, or 99% identical to amino acids 462-492 of SEQ ID NO:11. In some embodiments, the linker comprises amino acids 462-492 of SEQ ID NO:11.

In some embodiments, provided herein is a pharmaceutical composition comprising a therapeutically effective amount of a fusion antibody described herein and a pharmaceutically acceptable excipient.

In some embodiments, provided herein is an isolated polynucleotide encoding the fusion antibody described herein. In some embodiments, the isolated polynucleotide comprises the nucleic acid sequence of SEQ ID NO:15. In some embodiments, provided herein is a vector comprising an isolated polynucleotide provided herein. In some embodiments, provided herein is a vector comprising the nucleic acid sequence of SEQ ID NO:15. In some embodiments, provided herein is a host cell comprising a vector described herein. In some embodiments, the host cell is a Chinese Hamster Ovary (CHO) cell. In some aspects, provided herein is a method for treating an GLB1 deficiency in the central nervous system of a subject in need thereof, comprising systemically administering to the subject a therapeutically effective dose of a fusion antibody having GLB1 activity, wherein the fusion antibody comprises (a) a fusion protein comprising the amino acid sequence of an immunoglobulin heavy chain and an GLB1, and (b) an immunoglobulin light chain. In some embodiments, the amino acid sequence of the GLB1 is covalently linked to the carboxy terminus of the amino acid sequence of the immunoglobulin heavy chain. In some embodiments, provided herein is a method for treating an GLB1 deficiency in the central nervous system of a subject in need thereof, comprising systemically administering to the subject a therapeutically effective dose of a fusion antibody having GLB1 activity, wherein the fusion antibody comprises (a) a fusion protein comprising the amino acid sequence of an immunoglobulin light chain and an GLB1, and (b) an immunoglobulin heavy chain. In some embodiments, the amino acid sequence of the GLB1 is covalently linked to the carboxy terminus of the amino acid sequence of the immunoglobulin light chain. In some embodiments, the fusion antibody binds to the extracellular domain of an endogenous BBB receptor. In some embodiments, the endogenous BBB receptor is the human insulin receptor. In some embodiments, the fusion antibody is an antibody that binds to the endogenous BBB receptor. In some embodiments, the fusion antibody is an antibody that binds to the human insulin receptor. In some embodiments, the fusion antibody catalyzes degradation of GM1 gangliosides.

In certain embodiments, provided herein are methods and compositions for treating a subject suffering from an enzyme deficiency in the CNS. In certain embodiments, the methods provided herein comprise delivery of an enzyme deficient in GM1 to the CNS by systemically administering a therapeutically effective amount of a bifunctional fusion antibody or protein. In certain embodiments, the bifunctional fusion antibody comprises the amino acid sequences of an antibody to an endogenous blood brain barrier (BBB) receptor and an enzyme deficient in GM1. In some embodiments, the bifunctional fusion antibody is a human insulin antibody (HIR Ab) genetically fused to the enzyme. In certain embodiments, the fusion antibody binds to the extracellular domain of the insulin receptor and is transported across the BBB into the CNS, while retaining enzyme activity. In certain embodiments, the fusion antibody binds to the endogenous insulin receptor on the BBB, and acts as a molecular Trojan horse to ferry the enzyme into the brain. In certain embodiments, therapeutically effective systemic dose of a fusion antibody for systemic administration is based, in part, on the specific CNS uptake characteristics of the fusion antibody from peripheral blood as described herein.

In one aspect provided herein is a method for treating an enzyme deficiency in the central nervous system of a subject in need thereof, comprising systemically administering to the subject a therapeutically effective dose of a fusion antibody comprising the amino acid sequence of an immunoglobulin heavy chain, the amino acid sequence of an enzyme therapeutic in GM1, and the amino acid sequence of an immunoglobulin light chain. In some embodiments, the fusion antibody binds to an extracellular domain of an endogenous BBB receptor (e.g., the human insulin receptor). In some embodiments, the amino acid sequence of the enzyme is covalently linked to the carboxy terminus of the amino acid sequence of the immunoglobulin heavy chain.

In certain embodiments, the enzyme therapeutic in GM1 is a lysosomal enzyme.

In some embodiments, the enzyme therapeutic in GM1 is galactosidase beta-1 (GLB1).

In some embodiments, the fusion antibody catalyzes degradation of GM1 gangliosides.

In some embodiments, the enzyme retains at least 20% of its activity compared to its activity as a separate entity. In some embodiments, the enzyme and the immunoglobulin each retains at least 20% of its activity compared to its activity as a separate entity.

In some embodiments, at least about 600 ug of the enzyme are delivered to the brain. In some embodiments at least about 900 ug of the enzyme are delivered to the brain. In some embodiments at least about 1200 ug of the enzyme are delivered to the brain. In some embodiments at least about 2000 ug of the enzyme are delivered to the brain. In some embodiments at least about 3000 ug of the enzyme are delivered to the brain. In some embodiments at least about 4000 ug of the enzyme are delivered to the brain. In some embodiments at least about 5000 ug of the enzyme are delivered to the brain. In some embodiments at least about 8000 ug of the enzyme are delivered to the brain. In some embodiments at least about 10,000 ug of the enzyme are delivered to the brain. In some embodiments at least about 300 ug of the enzyme are delivered to the brain. In some embodiments at least about 100 ug of the enzyme are delivered to the brain. In some embodiments at least about 30 ug of the enzyme are delivered to the brain. In some embodiments at least about 10 ug of the enzyme are delivered to the brain. In some embodiments at least about 3 ug of the enzyme are delivered to the brain. In some embodiments at least about 1 ug of the enzyme are delivered to the brain.

In some embodiments, at least about 1500 ug of the enzyme are delivered to the brain, normalized per 50 kg body weight. In some embodiments, at least about 2250 ug of the enzyme are delivered to the brain, normalized per 50 kg body weight. In some embodiments, at least about 3000 ug of the enzyme are delivered to the brain, normalized per 50 kg body weight. In some embodiments, at least about 5000 ug of the enzyme are delivered to the brain, normalized per 50 kg body weight. In some embodiments, at least about 7500 ug of the enzyme are delivered to the brain, normalized per 50 kg body weight. In some embodiments, at least about 10,000 ug of the enzyme are delivered to the brain, normalized per 50 kg body weight. In some embodiments, at least about 15,000 ug of the enzyme are delivered to the brain, normalized per 50 kg body weight. In some embodiments, at least about 20,000 ug of the enzyme are delivered to the brain, normalized per 50 kg body weight. In some embodiments, at least about 25,000 ug of the enzyme are delivered to the brain, normalized per 50 kg body weight. In some embodiments, at least about 750 ug of the enzyme are delivered to the brain, normalized per 50 kg body weight. In some embodiments, at least about 250 ug of the enzyme are delivered to the brain, normalized per 50 kg body weight. In some embodiments, at least about 75 ug of the enzyme are delivered to the brain, normalized per 50 kg body weight. In some embodiments, at least about 25 ug of the enzyme are delivered to the brain, normalized per 50 kg body weight. In some embodiments, at least about 7.5 ug of the enzyme are delivered to the brain, normalized per 50 kg body weight. In some embodiments, at least about 2.5 ug of the enzyme are delivered to the brain, normalized per 50 kg body weight.

In some embodiments, the therapeutically effective dose of the fusion antibody comprises at least about 0.5 mg/Kg of body weight. In some embodiments, the therapeutically effective dose of the fusion antibody comprises at least about 0.6 mg/Kg of body weight. In some embodiments, the therapeutically effective dose of the fusion antibody comprises at least about 0.7 mg/Kg of body weight. In some embodiments, the therapeutically effective dose of the fusion antibody comprises at least about 0.8 mg/Kg of body weight. In some embodiments, the therapeutically effective dose of the fusion antibody comprises at least about 0.9 mg/Kg of body weight. In some embodiments, the therapeutically effective dose of the fusion antibody comprises at least about 1 mg/Kg of body weight. In some embodiments, the therapeutically effective dose of the fusion antibody comprises at least about 3 mg/Kg of body weight. In some embodiments, the therapeutically effective dose of the fusion antibody comprises at least about 6 mg/Kg of body weight. In some embodiments, the therapeutically effective dose of the fusion antibody comprises at least about 10 mg/Kg of body weight. In some embodiments, the therapeutically effective dose of the fusion antibody comprises at least about 50 mg/Kg of body weight. In some embodiments, the therapeutically effective dose of the fusion antibody comprises at least about 0.4 mg/Kg of body weight. In some embodiments, the therapeutically effective dose of the fusion antibody comprises at least about 0.3 mg/Kg of body weight. In some embodiments, the therapeutically effective dose of the fusion antibody comprises at least about 0.2 mg/Kg of body weight. In some embodiments, the therapeutically effective dose of the fusion antibody comprises at least about 0.1 mg/Kg of body weight.

In some embodiments, the enzyme specific activity of the fusion antibody is at least 20,000 units/mg protein. In some embodiments, the enzyme specific activity of the fusion antibody is at least 60,000 units/mg. In some embodiments, the enzyme specific activity of the fusion antibody is at least 120,000 units/mg. In some embodiments, the enzyme specific activity of the fusion antibody is at least 200,000 units/mg. In some embodiments, the enzyme specific activity of the fusion antibody is at least 500,000 units/mg. In some embodiments, the enzyme specific activity of the fusion antibody is at least 1,000,000 units/mg. In some embodiments, the enzyme specific activity of the fusion antibody is at least 5,000,000 units/mg.

In some embodiments, the enzyme deficiency in the central nervous system is GM1.

In some aspects, provided herein is a method for treating an enzyme deficiency in the central nervous system of a subject in need thereof, comprising systemically administering to the subject a therapeutically effective dose of a fusion antibody comprising (a) a fusion protein comprising the amino acid sequences of an immunoglobulin light chain and an enzyme deficient in GM1, and (b) an immunoglobulin heavy chain; wherein the fusion antibody crosses the blood brain barrier (BBB). In some embodiments, the amino acid sequence of the enzyme is covalently linked to the carboxy terminus of the amino acid sequence of the immunoglobulin heavy chain.

In some aspects, provided herein is a method for treating an enzyme deficiency in the central nervous system of a subject in need thereof, comprising systemically administering to the subject a therapeutically effective dose of a fusion antibody comprising (a) a fusion protein comprising an amino acid sequence that is at least 90% identical to SEQ ID NO:11; and (b) an immunoglobulin heavy chain. In some embodiments, the fusion antibody binds to an extracellular domain of an endogenous BBB receptor. In some embodiments, the endogenous BBB receptor is the human insulin receptor. In some embodiments, the fusion antibody catalyzes degradation of GM1 gangliosides. In some embodiments, the fusion protein comprises an amino acid sequence that is at least 95% identical to SEQ ID NO: 11. In some embodiments, the fusion protein comprises the amino acid sequence of SEQ ID NO: 11.

In some aspects, provided herein is a fusion antibody comprising (a) a fusion protein comprising an amino acid sequence that is at least 90% identical to SEQ ID NO: 11, and (b) an immunoglobulin heavy chain. In some embodiments, the fusion antibody binds to an extracellular domain of an endogenous BBB receptor. In some embodiments, the endogenous BBB receptor is the human insulin receptor. In some embodiments, the fusion antibody is an antibody that binds to the endogenous BBB receptor. In some embodiments, the fusion antibody is an antibody that binds to the human insulin receptor. In some embodiments, the fusion antibody catalyzes degradation of GM1 gangliosides. In some embodiments, the fusion protein comprises an amino acid sequence that is at least 95% identical to SEQ ID NO: 11. In some embodiments, the fusion protein comprises the amino acid sequence of SEQ ID NO: 11. In some embodiments, described herein are isolated polypeptides comprising an amino acid sequence at least 80%, 85%, 90%, 95%, 96%, 97%, 98%, or 99% identical to SEQ ID NO: 11. In some embodiments, described herein are isolated polypeptides comprising SEQ ID NO:11. In some embodiments, described herein are isolated polypeptides comprising amino acids 462-492 of SEQ ID NO:11.

In some aspects, provided herein is a fusion antibody comprising (a) a fusion protein comprising the amino acid sequence of an immunoglobulin light chain and an enzyme deficient in GM1, and (b) an immunoglobulin heavy chain. In some embodiments, the amino acid sequence of the enzyme is covalently linked to the carboxy terminus of the amino acid sequence of the immunoglobulin light chain.

In some embodiments, provided herein is a fusion antibody comprising (a) a fusion protein comprising the amino acid sequence of an immunoglobulin heavy chain and an enzyme deficient in GM1, and (b) an immunoglobulin light chain. In some embodiments, the amino acid sequence of the enzyme is covalently linked to the carboxy terminus of the amino acid sequence of the immunoglobulin heavy chain. In some embodiments, the fusion antibody binds to the extracellular domain of an endogenous BBB receptor. In some embodiments, the endogenous BBB receptor is the human insulin receptor. In some embodiments, the fusion antibody is an antibody that binds to the endogenous BBB receptor. In some embodiments, the fusion antibody is an antibody that binds to the human insulin receptor. In some embodiments, the fusion antibody catalyzes degradation of GM1 gangliosides.

In some embodiments, the fusion protein provided herein further comprises a linker between the amino acid sequence of the enzyme and the carboxy terminus of the amino acid sequence of the immunoglobulin heavy chain.

In some embodiments, provided herein is a pharmaceutical composition comprising a therapeutically effective amount of a fusion antibody described herein and a pharmaceutically acceptable excipient.

In some embodiments, provided herein is an isolated polynucleotide encoding the fusion antibody described herein. In some embodiments, the isolated polynucleotide comprises the nucleic acid sequence of SEQ ID NO:15. In some embodiments, provided herein is a vector comprising an isolated polynucleotide provided herein. In some embodiments, provided herein is a vector comprising the nucleic acid sequence of SEQ ID NO: 15. In some embodiments, provided herein is a host cell comprising a vector described herein. In some embodiments, the host cell is a Chinese Hamster Ovary (CHO) cell.

In some aspects, provided herein is a method for treating an enzyme deficiency in the central nervous system of a subject in need thereof, comprising systemically administering to the subject a therapeutically effective dose of a fusion antibody comprising (a) a fusion protein comprising the amino acid sequence of an immunoglobulin heavy chain and an enzyme deficient in GM1, and (b) an immunoglobulin light chain. In some embodiments, the amino acid sequence of the enzyme is covalently linked to the carboxy terminus of the amino acid sequence of the immunoglobulin heavy chain. In some embodiments, provided herein is a method for treating an enzyme deficiency in the central nervous system of a subject in need thereof, comprising systemically administering to the subject a therapeutically effective dose of a fusion antibody comprising (a) a fusion protein comprising the amino acid sequence of an immunoglobulin light chain and an enzyme deficient in GM1, and (b) an immunoglobulin heavy chain. In some embodiments, the amino acid sequence of the enzyme is covalently linked to the carboxy terminus of the amino acid sequence of the immunoglobulin heavy chain. In some embodiments, the fusion antibody binds to the extracellular domain of an endogenous BBB receptor. In some embodiments, the endogenous BBB receptor is the human insulin receptor. In some embodiments, the fusion antibody is an antibody that binds to the endogenous BBB receptor. In some embodiments, the fusion antibody is an antibody that binds to the human insulin receptor. In some embodiments, the fusion antibody catalyzes degradation of GM1 gangliosides.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the present embodiments are set forth with particularity in the appended claims. A better understanding of the features and advantages of the present embodiments will be obtained by reference to the following detailed description that sets forth illustrative embodiments, in which the principles of the present embodiments are utilized, and the accompanying drawings, as follow:

FIG. 1. Schematic depiction of a “molecular trojan horse” strategy in which the fusion antibody comprises an antibody to the extracellular domain of an endogenous BBB receptor (R), which acts as a molecular Trojan horse (TH), and GLB1, a lysosomal enzyme (E). Once inside brain cells, behind the BBB, the GLB1 part of the fusion antibody converts enzyme substrate (S) to product (P) and causes the degradation of GM1 gangliosides or keratan sulfate.

FIG. 2. An exemplary HIR Ab-GLB1 fusion antibody is formed by fusion of the amino terminus of the mature GLB1 to the carboxyl terminus of the heavy chain of the HIR Ab with an amino acid linker between the constant domain of the heavy chain (CL) and the GLB1 enzyme. The variable region of the light chain (VL) and the heavy chain (VH) is shown. The CH1, CH2, and CH3 constant domains of the heavy chain, and the constant domain of the light chain (CL) are shown.

FIG. 3. Agarose gel electrophoresis of pUC57-human GLB1 (minus the signal peptide) digested with StuI and HindIII. The human GLB1 synthetic gene (SEQ ID NO 12) was synthesized by a commercial vendor and provided in the pUC57 cloning vector. The human GLB1 cDNA is flanked by StuI and HindIII restriction endonuclease sites, respectively. The ˜2.0 kb human GLB1 engineered cDNA was released and separated from the ˜3.0 kb pUC57 plasmid backbone with StuI-HindIII digestion (lanes 2-4 are replicates) and isolated by agarose gel electrophoresis. Lane 1 is DNA standards.

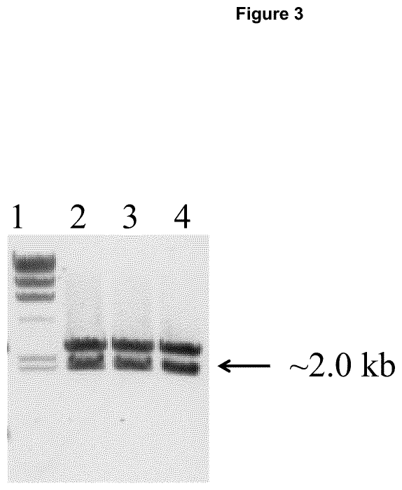

FIG. 4 Genetic engineering of HIR Ab HC-GLB1 expression vector. The HIR Ab-GLB1 heavy chain (HC) fusion protein expression vector, clone pHIR Ab-HC-GLB1, was engineered by insertion of the human GLB1 (minus the signal peptide) cDNA obtained by digestion of the pUC57-human GLB1 digested with StuI and HindIII and isolated by agarose gel electrophoresis (FIG. 3) into the HpaI-HindIII restriction endonuclease sites of the expression vector, designated pHIR Ab-HC. The latter contains either a 4-amino acid linker, Ser-Ser-Ser-Ser, or a 31-amino acid linker (SSSELKTPLGDTTHTSPRSPAPEFLGGPSSS) followed by the HpaI site. The HIR Ab HC-GLB1 cDNA encodes a fusion protein that is comprised of the amino acids 1-461 amino acids of the HIR Ab HC (SEQ ID NO:7) fused to the amino terminus of the 654 amino acids of mature GLB1, without the signal peptide (SEQ ID NO:9), via a 4 or a 31 amino acid linker. CMV=cytomegalovirus; BGH=bovine growth hormone; SV=simian virus; amp=ampicillin resistance; neo=neomycin; ori=origin of replication; DHFR=dihydrofolate reductase; LC=light chain; HC=heavy chain; GLB1=galactosidase beta-1.

FIG. 5. Amino acid sequence of an immunoglobulin heavy chain variable region from an exemplary human insulin receptor antibody directed against the extracellular domain of the human insulin receptor. The underlined sequences are a signal peptide, CDR1, CDR2, and CDR3, respectively. The heavy chain constant region, derived from human IgG1, is shown in italics.

FIG. 6. Amino acid sequence of an immunoglobulin light chain variable region from an exemplary human insulin receptor antibody directed against the extracellular domain of the human insulin receptor. The underlined sequences are a signal peptide, CDR1, CDR2, and CDR3, respectively. The constant region, derived from human kappa light chain, is shown in italics.

FIG. 7. A table showing the CDR1, CDR2, and CDR3 amino acid sequences from a heavy and light chain of an exemplary human insulin receptor antibody directed against the extracellular domain of the human insulin receptor.

FIG. 8. Amino acid sequence of GLB1 (NP_000395), not including the 23 amino acid enzyme signal peptide.

FIG. 9. Amino acid sequence of a fusion of an exemplary human insulin receptor antibody heavy chain to mature human GLB1. The underlined sequences are, in order, an IgG signal peptide, CDR1, CDR2, CDR3, and a 4-amino acid sequence linking the carboxy terminus of the heavy chain to the amino terminus of the mature GLB1. Sequence in italics corresponds to the heavy chain constant region, derived from human IgG1. The sequence in bold corresponds to human GLB1, minus the 23 amino acid signal peptide of the enzyme.

FIG. 10. Amino acid sequence of a fusion of an exemplary human insulin receptor antibody heavy chain to mature human GLB1. The underlined sequences are, in order, an IgG signal peptide, CDR1, CDR2, CDR3, and a 31-amino acid sequence linking the carboxy terminus of the heavy chain to the amino terminus of the mature GLB1. Sequence in italics corresponds to the heavy chain constant region, derived from human IgG1. The sequence in bold corresponds to human GLB1, minus the 23 amino acid signal peptide of the enzyme.

FIG. 11. Reducing SDS-PAGE of molecular weight (MW) standards, the purified HIR Ab (lane 1), and the purified HIR Ab-LL-GLB1 fusion protein (lane 2). Based on the relative migration of the MW standards, the HIR Ab is formed by a 56 kDa heavy chain (HC) and a 27 kDa light chain (LC).

The HIR Ab-LL-GLB1 fusion protein is formed by a 27 kDa HIR Ab light chain (HC) and a 140 kDa fusion protein of the HIR Ab heavy chain, mature GLB1, and the 31-amino acid (LL) linker joining the GLB1 to the C-terminus of the HIR Ab heavy chain.

FIG. 12. Western blot with either anti-human IgG primary antibody (A) or anti-human GLB1 primary antibody (B). In panel A, the immunoreactivity against the anti-human IgG primary antibody is compared for the HIR Ab (lane 1) and the HIR Ab-GLB1 fusion protein (lane 2). In panel B, the immunoreactivity against the anti-human GLB1 primary antibody is compared for the HIR Ab (lane 1) and the HIR Ab-GLB1 fusion protein (lane 2). Panel A shows the HIR Ab-GLB1 fusion protein and the HIR Ab have identical light chains on the anti-IgG Western. The fusion heavy chain of the HIR Ab-GLB1 fusion protein reacts with both the anti-IgG (lane 2, panel A) and the anti-human GLB1 antibody (lane 2, panel B).

FIG. 13. Binding of either the chimeric HIR Ab or the HIR Ab-LL-GLB1 fusion protein to the HIR extracellular domain (ECD) is saturable. The ED₅₀ of HIR Ab-LL-GLB1 binding to the HIR ECD is 119±18 ng/mL, which is 0.36±0.05 nM, based on a MW of 334 kDa determined by SDS-PAGE (FIG. 11). This is comparable to the ED₅₀ of the binding of the chimeric HIR Ab, 37±6 ng/mL, which is 0.25±0.04 nM, based on a MW of 150 kDa. The HIR Ab-LL-GLB1 fusion protein incorporates the 31-amino acid linker between the C-terminus of the HIR Ab heavy chain and the N-terminus of the mature GLB1.

FIG. 14. (A) The structure of the substrate of the GLB1 fluorometric enzyme assay, 4-methylumbelliferyl β-D-galactopyranoside (MUGP) is shown in panel A. Following cleavage of the molecule by GLB1, the substrate is converted to the fluorescent product, 4-methylumbelliferone (MU). (B) Linear formation of the MU product with respect to mass of HIR Ab-GLB1 fusion protein, with a fixed incubation time of 20 min. The GLB1 enzyme activity of the HIR Ab-GLB1 with either the 4-amino acid or the 31-amino acid linker is compared.

### DETAILED DESCRIPTION OF THE INVENTION

The blood brain barrier (BBB) is a severe impediment to the delivery of systemically administered lysosomal enzyme (e.g., recombinant GLB1) to the central nervous system. The methods and compositions described herein address the factors that are important in delivering a therapeutically significant level of an enzyme deficient in GM1, such as GLB1, across the BBB to the CNS: 1) Modification of an enzyme deficient in GM1 to allow it to cross the BBB via transport on an endogenous BBB transporter; 2) the amount and rate of uptake of systemically administered modified enzyme into the CNS, via retention of enzyme activity following the modification required to produce BBB transport. Various aspects of the methods and compositions described herein address these factors, by (1) providing fusion antibodies comprising an enzyme (e.g., a protein having GLB1 activity) fused, with or without intervening sequence, to an immunoglobulin (heavy chain or light chain) directed against the extracellular domain of an endogenous BBB receptor, and (2) establishing therapeutically effective systemic doses of the fusion antibodies based on the uptake in the CNS and the specific activity. In some embodiments, the antibody to the endogenous BBB receptor is an antibody to the human insulin receptor (HIR Ab).

Accordingly, provided herein are compositions and methods for treating an enzyme (e.g., GLB1) deficiency in the central nervous system by systemically administering to a subject in need thereof a therapeutically effective dose of a bifunctional BBB receptor Ab-enzyme fusion antibody having enzyme activity and selectively binding to the extracellular domain of an endogenous BBB receptor transporter such as the human insulin receptor.

### Some Definitions

“Treatment” or “treating” as used herein includes achieving a therapeutic benefit and/or a prophylactic benefit. By therapeutic benefit is meant eradication or amelioration of the underlying disorder or condition being treated. For example, in an individual with GM1, therapeutic benefit includes partial or complete halting of the progression of the disorder, or partial or complete reversal of the disorder. Also, a therapeutic benefit is achieved with the eradication or amelioration of one or more of the physiological or psychological symptoms associated with the underlying condition such that an improvement is observed in the patient, notwithstanding the fact that the patient may still be affected by the condition. A prophylactic benefit of treatment includes prevention of a condition, retarding the progress of a condition (e.g., slowing the progression of a lysosomal storage disorder), or decreasing the likelihood of occurrence of a condition. As used herein, “treating” or “treatment” includes prophylaxis.

As used herein, the term “effective amount” can be an amount, which when administered systemically, is sufficient to effect beneficial or desired results in the CNS, such as beneficial or desired clinical results, or enhanced cognition, memory, mood, or other desired CNS results. An effective amount is also an amount that produces a prophylactic effect, e.g., an amount that delays, or prevents the onset of the appearance of a pathological or undesired condition or symptoms of a pathological or undesired condition. Such conditions include, but are not limited to, mental retardation, hearing loss, and neurodegeneration. An effective amount can be administered in one or more administrations. In terms of treatment, an “effective amount” of a composition provided herein is an amount that is sufficient to palliate, ameliorate, stabilize, reverse or slow the progression of a disorder, e.g., a neurological disorder.

An “effective amount” may be of any of the compositions provided herein used alone or in conjunction with one or more agents used to treat a disease or disorder. An “effective amount” of a therapeutic agent within the meaning of the present embodiments will be determined by a patient's attending physician or veterinarian. Such amounts are readily ascertained by one of ordinary skill in the art and will a therapeutic effect when administered in accordance with the present embodiments. Factors which influence what a therapeutically effective amount will be include, the enzyme specific activity of the fusion antibody administered, its absorption profile (e.g., its rate of uptake into the brain), time elapsed since the initiation of the disorder, and the age, physical condition, existence of other disease states, and nutritional status of the individual being treated. Additionally, other medication the patient may be receiving will affect the determination of the therapeutically effective amount of the therapeutic agent to administer.

A “subject” or an “individual,” as used herein, is an animal, for example, a mammal. In some embodiments a “subject” or an “individual” is a human. In some embodiments, the subject suffers from GM1.

In some embodiments, a pharmacological composition comprising a fusion antibody is “administered peripherally” or “peripherally administered.” As used herein, these terms refer to any form of administration of an agent, e.g., a therapeutic agent, to an individual that is not direct administration to the CNS, e.g., that brings the agent in contact with the non-brain side of the blood-brain barrier. “Peripheral administration,” as used herein, includes intravenous, intra-arterial, subcutaneous, intramuscular, intraperitoneal, transdermal, by inhalation, transbuccal, intranasal, rectal, oral, parenteral, sublingual, or trans-nasal.

A “pharmaceutically acceptable carrier” or “pharmaceutically acceptable excipient” herein refers to any carrier that does not itself induce the production of antibodies harmful to the individual receiving the composition. Such carriers are well known to those of ordinary skill in the art. A thorough discussion of pharmaceutically acceptable carriers/excipients can be found in Remington's Pharmaceutical Sciences, Gennaro, A R, ed., 20th edition, 2000: Williams and Wilkins PA, USA. Exemplary pharmaceutically acceptable carriers can include salts, for example, mineral acid salts such as hydrochlorides, hydrobromides, phosphates, sulfates, and the like; and the salts of organic acids such as acetates, propionates, malonates, benzoates, and the like. For example, compositions described herein may be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate. The infusion solution may include 0 to 10% dextrose.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (e.g., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid; as such, the basic chemical structure of such amino acid analogs generally includes an a carbon that is bound to a hydrogen, a carboxyl group, an amino group, and an R group, such as, homoserine, norleucine, methionine sulfoxide, methionine methyl sulfonium. Such analogs may have modified R groups (such as, norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid may be one that is separated from the nucleic acids that normally flank it or other nucleic acids or components (proteins, lipids, etc. . . . ) in a sample. In another example, a polypeptide is purified if it is substantially removed from or concentrated in its natural environment. Methods for purification and isolation of nucleic acids and proteins are well known in the art.

### The Blood Brain Barrier

In one aspect, provided herein are compositions and methods that utilize an enzyme deficient in GM1 (e.g., GLB1) fused to an immunoglobulin capable of crossing the blood brain barrier (BBB) via receptor-mediated transport on an endogenous BBB receptor/transporter. An exemplary endogenous transporter for targeting is the insulin receptor on the BBB. The BBB insulin receptor mediates the transport of circulating insulin into the brain, as well as certain peptidomimetic monoclonal antibodies (MAb) such as the HIRMAb or HIR Ab. Other endogenous transporters that might be targeted with either an endogenous ligand or a peptidomimetic MAb include the BBB transferrin receptor, the BBB insulin-like growth factor (IGF) receptor, the BBB leptin receptor, or the BBB low density lipoprotein (LDL) receptor. The compositions and methods are useful in transporting GLB1 from the peripheral blood and across the blood brain barrier into the CNS. As used herein, the “blood-brain barrier” refers to the barrier between the peripheral circulation and the brain and spinal cord which is formed by tight junctions within the brain capillary endothelial plasma membranes and creates an extremely tight barrier that restricts the transport of molecules into the brain; the BBB is so tight that it is capable of restricting even molecules as small as urea, molecular weight of 60 Da. The blood-brain barrier within the brain, the blood-spinal cord barrier within the spinal cord, and the blood-retinal barrier within the retina, are contiguous capillary barriers within the central nervous system (CNS), and are collectively referred to as the blood-brain barrier or BBB.

The BBB limits the development of new neurotherapeutics, diagnostics, and research tools for the brain and CNS. Most large molecule therapeutics such as recombinant proteins, antisense drugs, gene medicines, purified antibodies, or RNA interference (RNAi)-based drugs do not cross the BBB in pharmacologically significant amounts. While it is generally assumed that small molecule drugs can cross the BBB, in fact, <2% of all small molecule drugs are active in the brain owing to the lack transport across the BBB. A molecule must be lipid soluble and have a molecular weight less than 400 Daltons (Da) in order to cross the BBB in pharmacologically significant amounts, and the vast majority of small molecules do not have these dual molecular characteristics. Therefore, most potentially therapeutic, diagnostic, or research molecules do not cross the BBB in pharmacologically active amounts. So as to bypass the BBB, invasive transcranial drug delivery strategies are used, such as intracerebro-ventricular (ICV) infusion, intracerebral (IC) administration, and convection enhanced diffusion (CED). Transcranial drug delivery to the brain is expensive, invasive, and largely ineffective. The ICV route, also called the intra-thecal (IT) route, delivers GLB1 only to the ependymal or meningeal surface of the brain, not into brain parenchyma, which is typical for drugs given by the ICV route. The IC administration of an enzyme such as GLB1, only provides local delivery, owing to the very low efficiency of protein diffusion within the brain. Similarly, the CED route only provides local delivery in brain near the catheter tip, as drug penetration via diffusion is limited.

The methods described herein offer an alternative to these highly invasive and generally unsatisfactory methods for bypassing the BBB, allowing a functional GLB1 to cross the BBB from the peripheral blood into the CNS following systemic administration of an HIR Ab-GLB1 fusion antibody composition described herein. The methods described herein exploit the expression of insulin receptors (e.g., human insulin receptors) on the BBB to shuttle a desired bifunctional HIR Ab-GLB1 fusion antibody from peripheral blood into the CNS.

### Endogenous Receptors

Certain endogenous small molecules in blood, such as glucose or amino acids, are water soluble, yet are able to penetrate the BBB, owing to carrier-mediated transport (CMT) on certain BBB carrier systems. For example, glucose penetrates the BBB via CMT on the GLUT1 glucose transporter. Amino acids, including therapeutic amino acids such as L-DOPA, penetrate the BBB via CMT on the LAT1 large neutral amino acid transporter. Similarly, certain endogenous large molecules in blood, such as insulin, transferrin, insulin-like growth factors, leptin, or low density lipoprotein are able to penetrate the BBB, owing to receptor-mediated transcytosis (RMT) on certain BBB receptor systems. For example, insulin penetrates the BBB via RMT on the insulin receptor. Transferrin penetrates the BBB via RMT on the transferrin receptor. Insulin-like growth factors may penetrate the BBB via RMT on the insulin-like growth factor receptor. Leptin may penetrate the BBB via RMT on the leptin receptor. Low density lipoprotein may penetrate the BBB via transport on the low density lipoprotein receptor.

The BBB has been shown to have specific receptors, including insulin receptors, that allow the transport from the blood to the brain of several macromolecules. In particular, insulin receptors are suitable as transporters for the HIR Ab-GLB1 fusion antibodies described herein. The HIR-GLB1 fusion antibodies described herein bind to the extracellular domain (ECD) of the human insulin receptor.

Insulin receptors and their extracellular, insulin binding domain (ECD) have been extensively characterized in the art both structurally and functionally. See, e.g., Yip et al (2003), J Biol. Chem, 278(30):27329-27332; and Whittaker et al. (2005), J Biol Chem, 280(22):20932-20936. The amino acid and nucleotide sequences of the human insulin receptor can be found under GenBank accession No. NM_000208.

Antibodies that Bind to an Insulin Receptor-Mediated Transport System

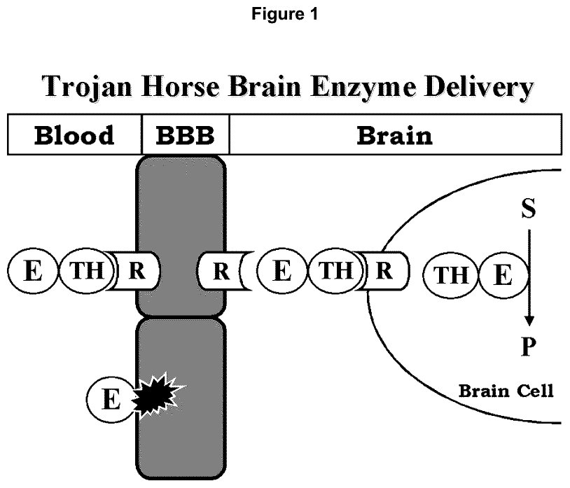

One noninvasive approach for the delivery of an enzyme deficient in GM1 (e.g., GLB1) to the CNS is to fuse the GLB1 to an antibody that selectively binds to the ECD of the insulin receptor. Insulin receptors expressed on the BBB can thereby serve as a vector for transport of the GLB1 across the BBB. Certain ECD-specific antibodies may mimic the endogenous ligand and thereby traverse a plasma membrane barrier via transport on the specific receptor system. Such insulin receptor antibodies act as molecular “Trojan horses,” or “TH” as depicted schematically in FIG. 1. By itself, GLB1 normally does not cross the blood-brain barrier (BBB). However, following fusion of the GLB1 to the TH, the enzyme is able to cross the BBB, and the brain cell membrane, by trafficking on the endogenous BBB receptor such as the IR, which is expressed at both the BBB and brain cell membranes in the brain (FIG. 1).

Thus, despite the fact that antibodies and other macromolecules are normally excluded from the brain, they can be an effective vehicle for the delivery of molecules into the brain parenchyma if they have specificity for the extracellular domain of a receptor expressed on the BBB, e.g., the insulin receptor. In certain embodiments, an HIR Ab-GLB1 fusion antibody binds an exofacial epitope on the human BBB HIR and this binding enables the fusion antibody to traverse the BBB via a transport reaction that is mediated by the human BBB insulin receptor.

The term “antibody” describes an immunoglobulin whether natural or partly or wholly synthetically produced. The term also covers any polypeptide or protein having a binding domain which is, or is homologous to, an antigen-binding domain. CDR grafted antibodies are also contemplated by this term.

“Native antibodies” and “native immunoglobulins” are usually heterotetrameric glycoproteins of about 150,000 daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“VH”) followed by a number of constant domains (“CH”). Each light chain has a variable domain at one end (“VL”) and a constant domain (“CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “variable domain” refers to protein domains that differ extensively in sequence among family members such as among different isoforms, or in different species. With respect to antibodies, the term “variable domain” refers to the variable domains of antibodies that are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the “framework region” or “FR”. The variable domains of unmodified heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from three “complementarity determining regions” or “CDRs”, which directly bind, in a complementary manner, to an antigen and are known as CDR1, CDR2, and CDR3 respectively.

In the light chain variable domain, the CDRs typically correspond to approximately residues 24-34 (CDRL1), 50-56 (CDRL2) and 89-97 (CDRL3), and in the heavy chain variable domain the CDRs typically correspond to approximately residues 31-35 (CDRH1), 50-65 (CDRH2) and 95-102 (CDRH3); Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol. 196:901 917 (1987)).

As used herein, “variable framework region” or “VFR” refers to framework residues that form a part of the antigen binding pocket or groove and/or that may contact antigen. In some embodiments, the framework residues form a loop that is a part of the antigen binding pocket or groove. The amino acids residues in the loop may or may not contact the antigen. In an embodiment, the loop amino acids of a VFR are determined by inspection of the three-dimensional structure of an antibody, antibody heavy chain, or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g. structural positions) can be less diversified. The three dimensional structure of the antibody variable domain can be derived from a crystal structure or protein modeling. In some embodiments, the VFR comprises, consist essentially of, or consists of amino acid positions corresponding to amino acid positions 71 to 78 of the heavy chain variable domain, the positions defined according to Kabat et al., 1991. In some embodiments, VFR forms a portion of Framework Region 3 located between CDRH2 and CDRH3. The VFR can form a loop that is well positioned to make contact with a target antigen or form a part of the antigen binding pocket.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2. The heavy-chain constant domains (Fc) that correspond to the different classes of immunoglobulins are called α, δ, ε, γ, and μ, respectively. The subunit structures and three-dimensional configurations of different classes of immunoglobulins are well known.

The “light chains” of antibodies (immunoglobulins) from any vertebrate species can be assigned to one of two clearly distinct types, called kappa or (“k”) and lambda or (“X”), based on the amino acid sequences of their constant domains.

In referring to an antibody or fusion antibody described herein, the terms “selectively bind,” “selectively binding,” “specifically binds,” or “specifically binding” refer to binding to the antibody or fusion antibody to its target antigen for which the dissociation constant (Kd) is about 10⁻⁶ M or lower, e.g., 10⁻⁷, 10⁻⁸, 10⁻⁹, 10⁻¹⁰, 10⁻¹¹, or 10⁻¹² M.

The term antibody as used herein will also be understood to mean one or more fragments of an antibody that retain the ability to specifically bind to an antigen, (see generally, Holliger et al., Nature Biotech. 23 (9) 1126-1129 (2005)). Non-limiting examples of such antibodies include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544 546), which consists of a VH domain; and (vi) an isolated complementarity determining region (CDR). Furthermore, although the two domains of the Fv fragment, VL and VH, are coded for by separate genes, they can be joined, using recombinant methods, by a synthetic or natural linker that enables them to be made as a single protein chain in which the VL and VH regions pair to form monovalent molecules (known as single chain Fv or scFv, or single chain Fab or scFab); see e.g., Bird et al. (1988) Science 242:423 426; and Huston et al. (1988) Proc. Natl. Acad. Sci. USA 85:5879 5883; and Osbourn et al. (1998) Nat. Biotechnol. 16:778). Such single chain antibodies are also intended to be encompassed within the term antibody. Any VH and VL sequences of specific single chain antibodies can be linked to human immunoglobulin constant region cDNA or genomic sequences, in order to generate expression vectors encoding complete IgG molecules or other isotypes. VH and VL can also be used in the generation of Fab, Fv or other fragments of immunoglobulins using either protein chemistry or recombinant DNA technology. Other forms of single chain antibodies, such as diabodies, or antibodies comprised of only a single monomeric variable domain, are also encompassed.

“F(ab′)2” and “Fab′” moieties can be produced by treating immunoglobulin (monoclonal antibody) with a protease such as pepsin and papain, and includes an antibody fragment generated by digesting immunoglobulin near the disulfide bonds existing between the hinge regions in each of the two H chains. For example, papain cleaves IgG upstream of the disulfide bonds existing between the hinge regions in each of the two H chains to generate two homologous antibody fragments in which an L chain composed of VL (L chain variable region) and CL (L chain constant region), and an H chain fragment composed of VH (H chain variable region) and CHγ1 (γ1 region in the constant region of H chain) are connected at their C terminal regions through a disulfide bond. Each of these two homologous antibody fragments is called Fab′. Pepsin also cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)2.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise a VH, a VL, or both a VH and VL domain of an antibody, wherein both domains are present in a single polypeptide chain. In some embodiments, the Fv polypeptide further comprises a polypeptide linker between the VH and VL domains which enables the sFv to form the desired structure for antigen binding. For a review of sFv see, e.g., Pluckthun in The Pharmacology of Monoclonal Antibodies, Vol. 113, Rosenburg and Moore eds. Springer-Verlag, New York, pp. 269 315 (1994).

A “chimeric” antibody includes an antibody derived from a combination of different mammals. The mammal may be, for example, a rabbit, a mouse, a rat, a goat, or a human. The combination of different mammals includes combinations of fragments from human and mouse sources.

In some embodiments, an antibody provided herein is a monoclonal antibody (MAb), typically a chimeric human-mouse antibody derived by humanization of a mouse monoclonal antibody. Such antibodies are obtained from, e.g., transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas.

For use in humans, a HIR Ab is preferred that contains enough human sequence that it is not significantly immunogenic when administered to humans, e.g., about 80% human and about 20% mouse, or about 85% human and about 15% mouse, or about 90% human and about 10% mouse, or about 95% human and 5% mouse, or greater than about 95% human and less than about 5% mouse, or 100% human. A more highly humanized form of the HIR MAb can also be engineered, and the humanized HIR Ab has activity comparable to the murine HIR Ab and can be used in embodiments provided herein. See, e.g., U.S. Patent Application Publication Nos. 20040101904, filed Nov. 27, 2002 and 20050142141, filed Feb. 17, 2005. Humanized antibodies to the human BBB insulin receptor with sufficient human sequences for use in the present embodiments are described in, e.g., Boado et al. (2007), Biotechnol Bioeng, 96(2):381-391.

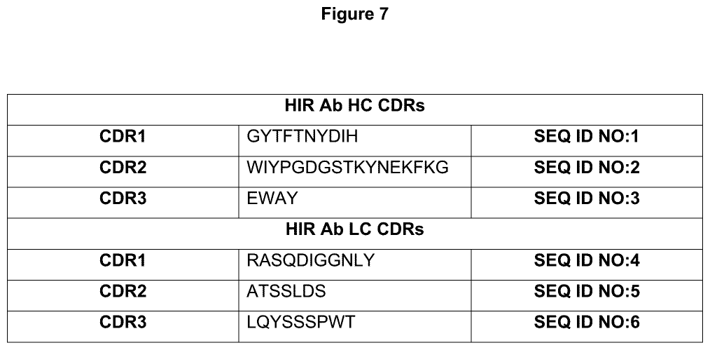

In exemplary embodiments, the HIR antibodies or fusion antibodies (e.g., HIR-GLB1) derived therefrom contain an immunoglobulin heavy chain comprising CDRs corresponding to the sequence of at least one of the HC CDRs listed in FIG. 7 (SEQ ID NOs 1-3) or a variant thereof. For example, a HC CDR1 corresponding to the amino acid sequence of SEQ ID NO:1 with up to 1, 2, 3, 4, 5, or 6 single amino acid mutations, a HC CDR2 corresponding to the amino acid sequence of SEQ ID NO:2 with up to 1, 2, 3, 4, 5, 6, 7, 8, 9, or 10 single amino acid mutations, or a HC CDR3 corresponding to the amino acid sequence of SEQ ID NO:3 with up to 1, or 2 single amino acid mutations, where the single amino acid mutations are substitutions, deletions, or insertions.

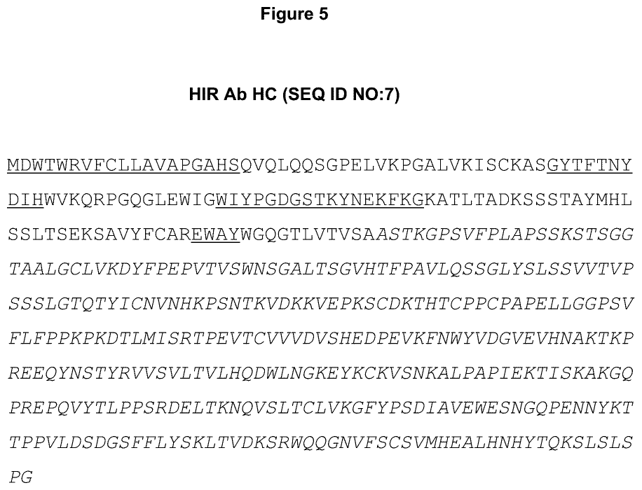

In other embodiments, the HIR Abs or fusion Abs (e.g., HIR Ab-GLB1) contain an immunoglobulin HC the amino acid sequence of which is at least 50% identical (e.g., at least, 55, 60, 65, 70, 75, 80, 85, 90, 95, or any other percent up to 100% identical) to SEQ ID NO:7 (shown in FIG. 5).

In some embodiments, the HIR Abs or fusion Abs (e.g., HIR Ab-GLB1) include an immunoglobulin light chain comprising CDRs corresponding to the sequence of at least one of the LC CDRs listed in FIG. 7 (SEQ ID NOs: 4-6) or a variant thereof. For example, a LC CDR1 corresponding to the amino acid sequence of SEQ ID NO:4 with up to 1, 2, 3, 4, or 5 single amino acid mutations, a LC CDR2 corresponding to the amino acid sequence of SEQ ID NO:5 with up to 1, 2, 3, or 4 single amino acid mutations, or a LC CDR3 corresponding to the amino acid sequence of SEQ ID NO:6 with up to 1, 2, 3, 4, or 5 single amino acid mutations.

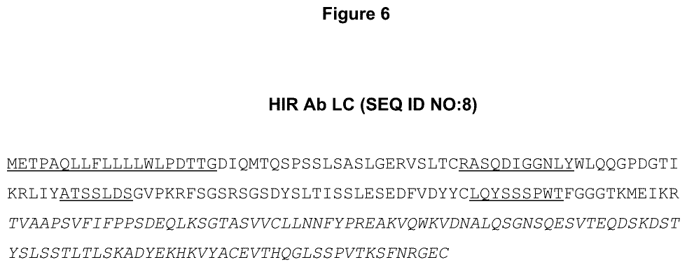

In other embodiments, the HIR Abs or fusion Abs (e.g., HIR Ab-GLB1) contain an immunoglobulin LC the amino acid sequence of which is at least 50% identical (e.g., at least, 55, 60, 65, 70, 75, 80, 85, 90, 95, or any other percent up to 100% identical) to SEQ ID NO:8 (shown in FIG. 6).

In yet other embodiments, the HIR Abs or fusion Abs (e.g., HIR Ab-GLB1) contain both a heavy chain and a light chain corresponding to any of the above-mentioned HIR heavy chains and HIR light chains.

HIR antibodies provided herein may be glycosylated or non-glycosylated. If the antibody is glycosylated, any pattern of glycosylation that does not significantly affect the function of the antibody may be used. Glycosylation can occur in the pattern typical of the cell in which the antibody is made, and may vary from cell type to cell type. For example, the glycosylation pattern of a monoclonal antibody produced by a mouse myeloma cell can be different than the glycosylation pattern of a monoclonal antibody produced by a transfected Chinese hamster ovary (CHO) cell. In some embodiments, the antibody is glycosylated in the pattern produced by a transfected Chinese hamster ovary (CHO) cell.

One of ordinary skill in the art will appreciate that current technologies permit a vast number of sequence variants of candidate HIR Abs or known HIR Abs to be readily generated be (e.g., in vitro) and screened for binding to a target antigen such as the ECD of the human insulin receptor or an isolated epitope thereof. See, e.g., Fukuda et al. (2006) “In vitro evolution of single-chain antibodies using mRNA display,” Nuc. Acid Res., 34(19) (published online) for an example of μltra high throughput screening of antibody sequence variants. See also, Chen et al. (1999), “In vitro scanning saturation mutagenesis of all the specificity determining residues in an antibody binding site,” Prot Eng, 12(4): 349-356. An insulin receptor ECD can be purified as described in, e.g., Coloma et al. (2000) Pharm Res, 17:266-274, and used to screen for HIR Abs and HIR Ab sequence variants of known HIR Abs.

Accordingly, in some embodiments, a genetically engineered HIR Ab, with the desired level of human sequences, is fused to an enzyme deficient in GM1 (e.g., GLB1), to produce a recombinant fusion antibody that is a bi-functional molecule. For example, the HIR Ab-GLB1 fusion antibody: (i) binds to an extracellular domain of the human insulin receptor, (ii) hydrolyze degradation of GM1 gangliosides; and (iii) is able to cross the BBB, via transport on the BBB HIR, and retain GLB1 activity once inside the brain, following peripheral administration.

### Galactosidase Beta-1 (GLB1)

Systemic administration (e.g., by intravenous injection) of recombinant GLB1 is not expected to rescue a deficiency of GLB1 in the CNS of patients suffering from GM1. GLB1 does not cross the BBB, and the lack of transport of the enzyme across the BBB prevents it from having a significant therapeutic effect in the CNS following peripheral administration. However, present inventors have discovered that when a lysosomal enzyme, such as GLB1, is fused to an antibody that crosses the BBB such as HIR Ab (e.g., by genetic fusion), this enzyme is now able to enter the CNS from blood following a non-invasive peripheral route of administration such as intravenous, intra-arterial, intramuscular, subcutaneous, intraperitoneal, or even oral administration. Administration of a HIR Ab-GLB1 fusion antibody enables delivery of GLB1 activity into the brain from peripheral blood. Described herein is the determination of a systemic dose of the HIR Ab-GLB1 fusion antibody that is therapeutically effective for treating a GLB1 deficiency in the CNS. As described herein, appropriate systemic doses of an HIR Ab-GLB1 fusion antibody are established based on a quantitative determination of CNS uptake characteristics and enzymatic activity of an HIR Ab-enzyme fusion antibody.

GM1 gangliosides are synthesized in the central nervous system as well as peripheral tissues. As used herein, GLB1 (e.g., the human GLB1 sequence listed under GenBank Accession No. NP_000395) refers to any naturally occurring or artificial enzyme that can catalyze the degradation of GM1 gangliosides.

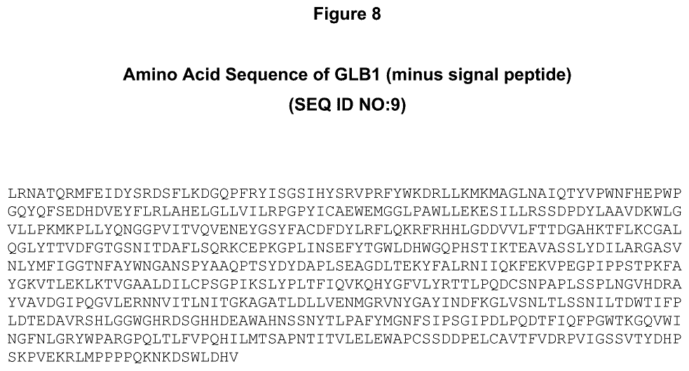

In some embodiments, GLB1 has an amino acid sequence that is at least 50% identical (e.g., at least, 55, 60, 65, 70, 75, 80, 85, 90, 95, or any other percent up to 100% identical) to the amino acid sequence of human GLB1, a 677 amino acid protein listed under Genbank NP_000395, or a 654 amino acid subsequence thereof, which lacks a 23 amino acid signal peptide, and corresponds to SEQ ID NO:9 (FIG. 8). The cloning and expression of human GLB1 has been described by Oshima et al (1988), “Cloning, sequencing, and expression of cDNA for human beta-galactosidase,” Biochem Biophys Res Comm 157: 238-244.

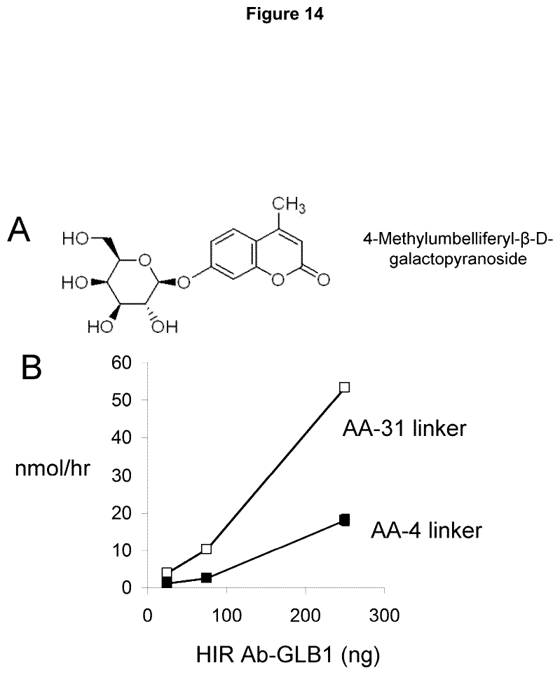

In some embodiments, GLB1 has an amino acid sequence at least 50% identical (e.g., at least, 55, 60, 65, 70, 75, 80, 85, 90, 95, or any other percent up to 100% identical) to SEQ ID NO:9 (shown in FIG. 8). Sequence variants of a canonical GLB1 sequence such as SEQ ID NO:9 can be generated, e.g., by random mutagenesis of the entire sequence or specific subsequences corresponding to particular domains. Alternatively, site directed mutagenesis can be performed reiteratively while avoiding mutations to residues known to be critical to GLB1 function such as those given above. Further, in generating multiple variants of an GLB1 sequence, mutation tolerance prediction programs can be used to greatly reduce the number of non-functional sequence variants that would be generated by strictly random mutagenesis. Various programs) for predicting the effects of amino acid substitutions in a protein sequence on protein function (e.g., SIFT, PolyPhen, PANTHER PSEC, PMUT, and TopoSNP) are described in, e.g., Henikoff et al. (2006), “Predicting the Effects of Amino Acid Substitutions on Protein Function,” Annu. Rev. Genomics Hum. Genet., 7:61-80. GLB1 sequence variants can be screened for of GLB1 activity/retention of GLB1 activity by a fluorometric enzymatic assay known in the art (Suzuki K. Globoid cell leukodystrophy (Krabe disease) and GM1 gangliosidosis. In: Glew R H, Peters S P (Editors), Practical enzymology of the sphingolipidoses. New York: Alan R. Liss; 1977. p 101-136), using as substrate 4-methylumbelliferyl β-D-galactopyranoside (MUGP), which is used in FIG. 14. Accordingly, one of ordinary skill in the art will appreciate that a very large number of operable GLB1 sequence variants can be obtained by generating and screening extremely diverse “libraries” of GLB1 sequence variants by methods that are routine in the art, as described above.

Percent sequence identity is determined by conventional methods. See, for example, Altschul et al., Bull. Math. Bio. 48:603 (1986), and Henikoff and Henikoff, Proc. Nal. Acad Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of another peptide. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO:9) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

The present embodiments also include proteins having a conservative amino acid change, compared with an amino acid sequence disclosed herein. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present embodiments. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

It also will be understood that amino acid sequences may include additional residues, such as additional N- or C-terminal amino acids, and yet still be essentially as set forth in one of the sequences disclosed herein, so long as the sequence retains sufficient biological protein activity to be functional in the compositions and methods of the present embodiments.

### Compositions

It has been found that the bifunctional fusion antibodies described herein, retain a high proportion of the activity of their separate constituent proteins, e.g., binding of the antibody capable of crossing the BBB (e.g., HIR Ab) to the extracellular domain of an endogenous receptor on the BBB (e.g., IR ECD), and the enzymatic activity of an enzyme deficient in GM1 (e.g., GLB1). Construction of cDNAs and expression vectors encoding any of the proteins described herein, as well as their expression and purification are well within those of ordinary skill in the art, and are described in detail herein in, e.g., Examples 1-3, and, in Boado et al (2007), Biotechnol Bioeng 96:381-391, U.S. patent application Ser. No. 11/061,956, and U.S. patent application Ser. No. 11/245,710.

Described herein are bifunctional fusion antibodies containing an antibody to an endogenous BBB receptor (e.g., HIR Ab), as described herein, capable of crossing the BBB fused to GLB1, where the antibody to the endogenous BBB receptor is capable of crossing the blood brain barrier and the GLB1 each retain an average of at least about 10, 20, 30, 40, 50, 60, 70, 80, 90, 95, 99, or 100% of their activities, compared to their activities as separate entities. In some embodiments, provided herein is a HIR Ab-GLB1 fusion antibody where the HIR Ab and GLB1 each retain an average of at least about 50% of their activities, compared to their activities as separate entities. In some embodiments, provided herein is a HIR Ab-GLB1 fusion antibody where the HIR Ab and GLB1 each retain an average of at least about 60% of their activities, compared to their activities as separate entities. In some embodiments, provided herein is a HIR Ab-GLB1 fusion antibody where the HIR Ab and GLB1 each retain an average of at least about 70% of their activities, compared to their activities as separate entities. In some embodiments, provided herein is a HIR Ab-GLB1 fusion antibody where the HIR Ab and GLB1 each retain an average of at least about 80% of their activities, compared to their activities as separate entities. In some embodiments, provided herein is a fusion HIR Ab-GLB1 fusion antibody where the HIR Ab and GLB1 each retain an average of at least about 90% of their activities, compared to their activities as separate entities. In some embodiments, the HIR Ab retains at least about 10, 20, 30, 40, 50, 60, 70, 80, 90, 95, 99, or 100% of its activity, compared to its activity as a separate entity, and the GLB1 retains at least about 10, 20, 30, 40, 50, 60, 70, 80, 90, 95, 99, or 100% of its activity, compared to its activity as a separate entity. Accordingly, described herein are compositions containing a bifunctional HIR Ab-GLB1 fusion antibody capable of crossing the BBB, where the constituent HIR Ab and GLB1 each retain, as part of the fusion antibody, an average of at least about 10, 20, 30, 40, 50, 60, 70, 80, 90, 95, 99, or 100% of their activities, e.g., HIR binding and GLB1 activity, respectively, compared to their activities as separate proteins. An HIR Ab GLB1 fusion antibody refers to a fusion protein comprising any of the HIR antibodies and GLB1 described herein.

In any of the embodiments provided herein, HIR Ab may be replaced by an antibody to an endogenous BBB receptor described herein, such as an antibody to transferrin receptor, leptin receptor, lipoprotein receptor, or the insulin-like growth factor (IGF) receptor, or other similar endogenous BBB receptor-mediated transport system.

In some cases, in the fusion antibodies described herein, the covalent linkage between the antibody and the GLB1 may be to the carboxy or amino terminal of the antibody heavy or light chain. In some cases, the covalent linkage between the antibody and the GLB1 is to the amino or carboxy terminal of the GLB1. Generally, the linkages provided herein permit the fusion antibody to bind to the ECD of a BBB receptor (e.g, IR) and cross the blood brain barrier, and allows the GLB1 to retain a therapeutically useful portion of its activity. In certain embodiments, the covalent link is between an HC of the antibody and the GLB1 or a LC of the antibody and the GLB1, or between the GLB1 and a single chain antibody. Any suitable linkage may be used, e.g., carboxy terminus of light chain to amino terminus of GLB1, carboxy terminus of heavy chain to amino terminus of GLB1, amino terminus of light chain to carboxy terminus of GLB1, amino terminus of heavy chain to carboxy terminus of GLB1, amino terminus of GLB1 to carboxy terminus of a single chain antibody, or carboxy terminus of GLB1 to amino terminus of single chain antibody. In some embodiments, the linkage is from the carboxy terminus of the LC to the amino terminus of the GLB1.

The GLB1 may be fused, or covalently linked, to the targeting antibody (e.g., MAb, HIR-MAb) through a linker. A linkage between terminal amino acids can be accomplished by an intervening peptide linker sequence that forms part of the fused amino acid sequence. The peptide sequence linker may be 0, 1, 2, 3, 4, 5, 6, 7, 8, 9, 10, 15, 20, or more than 20 amino acids in length. In some embodiments, including some preferred embodiments, the peptide linker is less than 30, 20, 15, 14, 13, 12, 11, 10, 9, 8, 7, 6, 5, 4, 3, 2, or 1 amino acids in length. In some embodiments, including some preferred embodiments, the peptide linker is at least 20 to 25 amino acids in length. In some embodiments, then peptide linker is 4-20 amino acids in length. In some embodiments, the peptide linker is 6-25 amino acids in length. In some embodiments, the peptide linker is 2-40, 3-40, 4-40, 2-35, 3-35, or 3-31 amino acids in length. In some embodiments, the peptide linker is 30-35 amino acids in length. In some embodiments, the linker is 30-33 amino acids in length. In some embodiments, the peptide linker is 30-40 amino acids in length. In some embodiments, the peptide linker is 30-35 amino acids in length. In some embodiments, the peptide linker is 20-60 amino acids in length. In some embodiments, the peptide linker is 21-59 amino acids in length. In some embodiments, the peptide linker is 22-58 amino acids in length. In some embodiments, the peptide linker is 23-58 amino acids in length. In some embodiments, the peptide linker is 20-25 amino acids in length. In some embodiments, the peptide linker is 55-60 amino acids in length. In some embodiments, the peptide linker is 3-60 amino acids in length. In some embodiments, the peptide linker is 4 amino acids in length. In some embodiments, the linker comprises amino acids 462-465 of SEQ ID NO:10. In some embodiments, the peptide linker is 31 amino acids in length. In some embodiments, the linker comprises amino acids 462-492 of SEQ ID NO:11. In some embodiments, the GLB1 is directly linked to the targeting antibody, and the peptide linker is therefore 0 amino acids in length.

In some embodiments, the linker comprises glycine, serine, and/or alanine residues in any combination or order. In some cases, the combined percentage of glycine, serine, and alanine residues in the linker is at least 5%, 10%, 15%, 20%, 25%, 30%, 40%, 50%, 60%, 70%, 75%, 80%, 90%, or 95% of the total number of residues in the linker. In some preferred embodiments, the combined percentage of glycine, serine, and alanine residues in the linker is at least 50%, 60%, 70%, 75%, 80%, 90%, or 95% of the total number of residues in the linker. In some embodiments, any number of combinations of amino acids (including natural or synthetic amino acids) can be used for the linker. In some embodiments, a three amino acid linker is used. In some embodiments, the linker has the sequence Ser-Ser-Ser. In some embodiments, a two amino acid linker comprises glycine, serine, and/or alanine residues in any combination or order (e.g., Gly-Gly, Ser-Gly, Gly-Ser, Ser-Ser. Ala-Ala, Ser-Ala, or Ala-Ser linker). In some embodiments, a two amino acid linker consists of one glycine, serine, and/or alanine residue along with another amino acid (e.g., Ser-X, where X is any known amino acid). In still other embodiments, the two-amino acid linker consists of any two amino acids (e.g., X-X), exept gly, ser, or ala.

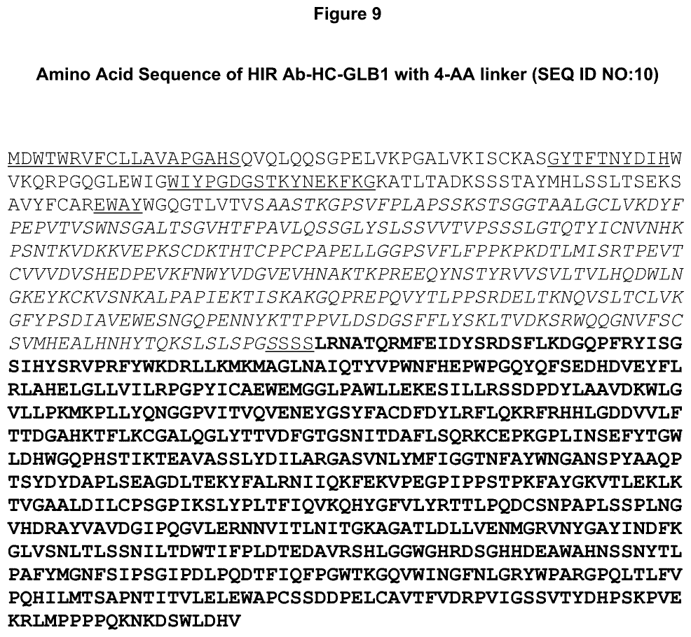

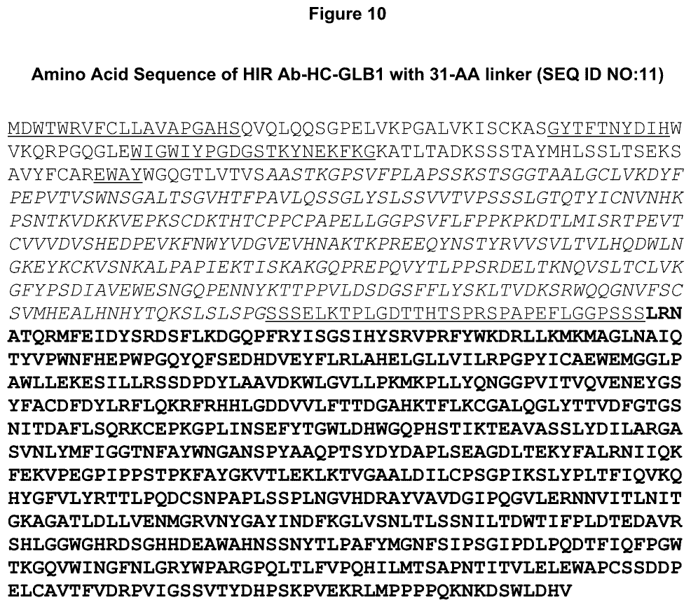

In some embodiments, the linker is comprised of 4 serine residues as shown in FIG. 9 (underlined), which corresponds to amino acids 462-465 of SEQ ID NO:10 (FIG. 9). In some embodiments, the linker is derived from the sequence of an endogenous human protein, such as the hinge region from human IgG3, which is comprised of 62 amino acids. In some embodiments, the linker is derived from a truncated version of the human IgG3 hinge region. In some embodiments, the cysteine residues of the human IgG3 hinge region are mutated to serine residues, so as to eliminate disulfide bonding between chains. In some embodiments, a serine-serine-serine spacer is placed on both the amino terminal and carboxyl terminal sides of the hinge sequence. A 31 AA linker includes 25 AA from the human IgG3 hinge region, which is derived from the 12 amino acids of the upper hinge region, followed by 5 amino acids of the first part of the core hinge region, followed by 8 amino acids of the lower hinge region, and is flanked by a Ser-Ser-Ser sequence on the amino terminus and a Ser-Ser-Ser sequence on the carboxyl terminus. The 2 cysteine residues of the first part of the core hinge region are mutated to serine residues, so as to eliminate disulfide bonding. These embodiments comprise the linker shown in FIG. 10 (underlined), which corresponds to amino acids 462-492 of SEQ ID NO:11 (FIG. 10).

As described herein, in some embodiments a linker that is greater than two amino acids in length. Such linker may also comprise glycine, serine, and/or alanine residues in any combination or order, as described further herein. In some embodiments, the linker consists of one glycine, serine, and/or alanine residue along with other amino acids (e.g., Ser-nX, where X is any known amino acid, and n is the number of amino acids). In still other embodiments, the linker consists of any two amino acids (e.g., X-X). In some embodiments, said any two amino acids are Gly, Ser, or Ala, in any combination or order, and within a variable number of amino acids intervening between them. In an example of an embodiment, the linker consists of at least one Gly. In an example of an embodiment, the linker consists of at least one Ser. In an example of an embodiment, the linker consists of at least one Ala. In some embodiments, the linker consists of at least 1, 2, 3, 4, 5, 6, 7, 8, 9, or 10 Gly, Ser, and/or Ala residues. In preferred embodiments, the linker comprises Gly and Ser in repeating sequences, in any combination or number, such as (Gly₄Ser)₃, or other variations.

A linker for use in the present embodiments may be designed by using any method known in the art. For example, there are multiple publicly-available programs for determining optimal amino acid linkers in the engineering of fusion proteins. Publicly-available computer programs (such as the LINKER program) that automatically generate the amino acid sequence of optimal linkers based on the user's input of the sequence of the protein and the desired length of the linker may be used for the present methods and compositions. Often, such programs may use observed trends of naturally-occurring linkers joining protein subdomains to predict optimal protein linkers for use in protein engineering. In some cases, such programs use other methods of predicting optimal linkers. Examples of some programs suitable for predicting a linker for the present embodiments are described in the art, see, e.g., Xue et al. (2004) Nucleic Acids Res. 32, W562-W565 (Web Server issue providing internet link to LINKER program to assist the design of linker sequences for constructing functional fusion proteins); George and Heringa, (2003), Protein Engineering, 15(11):871-879 (providing an internet link to a linker program and describing the rational design of protein linkers); Argos, (1990), J. Mol. Biol. 211:943-958; Arai et al. (2001) Protein Engineering, 14(8):529-532; Crasto and Feng, (2000) Protein Engineering 13(5):309-312.

The peptide linker sequence may include a protease cleavage site, however this is not a requirement for activity of the GLB1; indeed, an advantage of these embodiments is that the bifunctional HIR Ab-GLB1 fusion antibody, without cleavage, is partially or fully active both for transport and for activity once across the BBB. FIG. 10 shows an exemplary embodiment of the amino acid sequence of a HIR Ab-GLB1 fusion antibody (SEQ ID NO:11) in which the HC is fused through its carboxy terminus via a 31 amino acid linker to the amino terminus of the GLB1. In some embodiments, the fused GLB1 sequence is devoid of its 23 amino acid signal peptide, as shown in FIG. 8.

In some embodiments, a HIR Ab-GLB1 fusion antibody provided herein comprises both a HC and a LC. In some embodiments, the HIR Ab-GLB1 fusion antibody is a monovalent antibody. In other embodiments, the HIR Ab-GLB1 fusion antibody is a divalent antibody, as described herein in the Example section.

In some embodiments, the HIR Ab used as part of the HIR Ab-GLB1 fusion antibody can be glycosylated or nonglycosylated; in some embodiments, the antibody is glycosylated, e.g., in a glycosylation pattern produced by its synthesis in a CHO cell.

As used herein, “activity” includes physiological activity (e.g., ability to cross the BBB and/or therapeutic activity), binding affinity of the HIR Ab for the IR ECD, or the enzymatic activity of GLB1.

Transport of a HIR Ab-GLB1 fusion antibody across the BBB may be compared to transport across the BBB of the HIR Ab alone by standard methods. For example, pharmacokinetics and brain uptake of the HIR Ab-GLB1 fusion antibody by a model animal, e.g., a mammal such as a primate, may be used. Similarly, standard models for determining GLB1 activity may also be used to compare the function of the GLB1 alone and as part of a HIR Ab-GLB1 fusion antibody. See, e.g., Example 6, which demonstrates the enzymatic activity of GLB1 versus HIR Ab-GLB1 fusion antibody. Binding affinity for the IR ECD can be compared for the HIR Ab-GLB1 fusion antibody versus the HIR Ab alone. See, e.g., Example 6 herein.

Also included herein are pharmaceutical compositions that contain one or more HIR Ab-GLB1 fusion antibodies described herein and a pharmaceutically acceptable excipient. A thorough discussion of pharmaceutically acceptable carriers/excipients can be found in Remington's Pharmaceutical Sciences, Gennaro, A R, ed., 20th edition, 2000: Williams and Wilkins PA, USA. Pharmaceutical compositions of the present embodiments include compositions suitable for administration via any peripheral route, including intravenous, subcutaneous, intramuscular, intraperitoneal injection; oral, rectal, transbuccal, pulmonary, transdermal, intranasal, or any other suitable route of peripheral administration.

The compositions provided herein are particular suited for injection, e.g., as a pharmaceutical composition for intravenous, subcutaneous, intramuscular, or intraperitoneal administration. Aqueous compositions provided herein comprise an effective amount of a composition of the present embodiments, which may be dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, e.g., a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Exemplary pharmaceutically acceptable carriers for injectable compositions can include salts, for example, mineral acid salts such as hydrochlorides, hydrobromides, phosphates, sulfates, and the like; and the salts of organic acids such as acetates, propionates, malonates, benzoates, and the like. For example, compositions provided herein may be provided in liquid form, and formulated in saline, with or without added dextrose between 0 to 10%, based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate. Under ordinary conditions of storage and use, these preparations can contain a preservative to prevent the growth of microorganisms. The prevention of the action of microorganisms can be brought about by various antibacterial and antifungal agents, for example, parabens, chlorobutanol; phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate, and gelatin.

For human administration, preparations can meet sterility, pyrogenicity, general safety, and purity standards as required by FDA and other regulatory agency standards. The active compounds can generally be formulated for parenteral administration, e.g., formulated for injection via the intravenous, intramuscular, subcutaneous, intralesional, or intraperitoneal routes. The preparation of an aqueous composition that contains an active component or ingredient will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

In some cases, sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, methods of preparation may include vacuum-drying and freeze-drying techniques which yield a powder of the active ingredient plus any additional desired ingredient from a previously sterile-filtered solution thereof.

Upon formulation, solutions may be systemically administered in a manner compatible with the dosage formulation and in such amount as is therapeutically effective based on the criteria described herein. The formulations are easily administered in a variety of dosage forms, such as the type of injectable solutions described above, but drug release capsules and the like can also be employed

The appropriate quantity of a pharmaceutical composition to be administered, the number of treatments, and unit dose will vary according to the CNS uptake characteristics of a HIR Ab-GLB1 fusion antibody as described herein, and according to the subject to be treated, the state of the subject and the effect desired. The person responsible for administration may, in any event, determine the appropriate dose for the individual subject.

In addition to the compounds formulated for parenteral administration, such as intravenous or intramuscular injection, other alternative methods of administration of the present embodiments may also be used, including but not limited to intradermal administration (See U.S. Pat. Nos. 5,997,501; 5,848,991; and 5,527,288), pulmonary administration (See U.S. Pat. Nos. 6,361,760; 6,060,069; and 6,041,775), buccal administration (See U.S. Pat. Nos. 6,375,975; and 6,284,262), transdermal administration (See U.S. Pat. Nos. 6,348,210; and 6,322,808) and transmucosal administration (See U.S. Pat. No. 5,656,284). Such methods of administration are well known in the art. One may also use intranasal administration of the present embodiments, such as with nasal solutions or sprays, aerosols or inhalants. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and include, for example, antibiotics and antihistamines and are used for asthma prophylaxis.

Additional formulations, which are suitable for other modes of administration, include suppositories and pessaries. A rectal pessary or suppository may also be used. Suppositories are solid dosage forms of various weights and shapes, usually medicated, for insertion into the rectum or the urethra. After insertion, suppositories soften, melt or dissolve in the cavity fluids. For suppositories, traditional binders and carriers generally include, for example, polyalkylene glycols or triglycerides; such suppositories may be formed from mixtures containing the active ingredient in any suitable range, e.g., in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations, or powders. In certain defined embodiments, oral pharmaceutical compositions will comprise an inert diluent or assimilable edible carrier, or they may be enclosed in a hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations can contain at least 0.1% of active compound. The percentage of the compositions and preparations may, of course, be varied, and may conveniently be between about 2 to about 75% of the weight of the unit, or between about 25-60%. The amount of active compounds in such therapeutically useful compositions is such that a suitable dosage will be obtained.

The tablets, troches, pills, capsules and the like may also contain the following: a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent, methylene and propyl parabens as preservatives, a dye and flavoring, such as cherry or orange flavor. In some embodiments, an oral pharmaceutical composition may be enterically coated to protect the active ingredients from the environment of the stomach; enteric coating methods and formulations are well-known in the art.

### Methods

Described herein are methods for delivering an effective dose of an enzyme deficient in GM1 (e.g., GLB1) to the CNS across the BBB by systemically administering a therapeutically effective amount of a fusion antibody, as described herein. In some embodiments, the fusion antibody provided herein is a HIR Ab-GLB1. Suitable systemic doses for delivery of a HIR Ab-GLB1 fusion antibody is based on its CNS uptake characteristics and GLB1 specific activity as described herein. Systemic administration of a HIR Ab-GLB1 fusion antibody to a subject suffering from an GLB1 deficiency is an effective approach to the non-invasive delivery of GLB1 to the CNS.

The amount of a fusion antibody that is a therapeutically effective systemic dose of a fusion antibody depends, in part, on the CNS uptake characteristics of the fusion antibody to be administered, as described herein, e.g., the percentage of the systemically administered dose to be taken up in the CNS.

In some embodiments, 1% (e.g., about 0.3%, 0.4%, 0.48%, 0.6%, 0.74%, 0.8%, 0.9%, 1.05, 1.1, 1.2, 1.3%, 1.5%, 2%, 2.5%, 3%, or any % from about 0.3% to about 3%) of the systemically administered HIR Ab-GLB1 fusion antibody is delivered to the brain as a result of its uptake from peripheral blood across the BBB. In some embodiments, at least 0.5%, (e.g., about 0.3%, 0.4%, 0.48%, 0.6%, 0.74%, 0.8%, 0.9%, 1.05, 1.1, 1.2, 1.3%, 1.5%, 2%, 2.5%, 3%, or any % from about 0.3% to about 3%) of the systemically administered dose of the HIR Ab-GLB1 fusion antibody is delivered to the brain within two hours or less, e.g., 1.8, 1.7, 1.5, 1.4, 1.3, 1.2, 1.1, 0.9, 0.8, 0.6, 0.5 or any other period from about 0.5 to about two hours after systemic administration.

Accordingly, in some embodiments provided herein are methods of administering a therapeutically effective amount of a fusion antibody described herein systemically, to a 5 to 50 kg human, such that the amount of the fusion antibody to cross the BBB provides at least 0.01 ng of GLB1 protein/mg protein in the subject's brain, e.g., 0.03, 0.1, 0.3, 1, 3, 10, 30, 100 or 300 or any other value from 0.01 to 300 ng of GLB1 protein/mg protein in the subject's brain.

In some embodiments, the total number of units of enzyme (e.g., GLB1) activity delivered to a subject's brain is at least, 0.2 units per gram brain, e.g., at least 1, 3, 10, 30, 100, 300, 1000, 3000, or 6000 or any other total number of GLB1 units from about 0.2 to 6000 units of GLB1 activity delivered per gram brain.

In some embodiments, a therapeutically effective systemic dose comprises at least 100, 300, 1000, 3000, 10000, 30000, 100,000, 300,000, 1,000,000, 3,000,000, 10,000,000, or 30,000,000 or any other systemic dose from about 100 to 30,000,000 units of enzyme (e.g., GLB1) activity.

In other embodiments, a therapeutically effective systemic dose is at least about 10, 30, 100, 300, 1,000, 3,000, 10,000, 30,000, 100,000, 300,000, 1,000,000, or 3,000,0000 or any other number of units from about 10 to 3,000,000 units of enzyme activity/kg of body weight.

One of ordinary skill in the art will appreciate that the mass amount of a therapeutically effective systemic dose of a fusion antibody provided herein will depend, in part, on its enzyme (e.g., GLB1) specific activity. In some embodiments, the specific activity of a fusion antibody is at least 20,000 U/mg of protein, at least about 100,000, 300,000, 1,000,000, or 5,000,000, or any other specific activity value from about 20,000 units/mg to about 5,000,000 units/mg.

Thus, with due consideration of the specific activity of a fusion antibody provided herein and the body weight of a subject to be treated, a systemic dose of the fusion antibody can be at least 1 mg, e.g., 3, 10, 30, 100, 300, or 1000, or any other value from about 1 mg to about 1000 mg of fusion antibody (e.g., HIR Ab-GLB1).

The term “systemic administration” or “peripheral administration,” as used herein, includes any method of administration that is not direct administration into the CNS, e.g., that does not involve physical penetration or disruption of the BBB. “Systemic administration” includes, but is not limited to, intravenous, intra-arterial intramuscular, subcutaneous, intraperitoneal, intranasal, transbuccal, transdermal, rectal, transalveolar (inhalation), or oral administration. Any suitable fusion antibody, as described herein, may be used.

A GLB1 deficiency as referred to herein includes, one or more conditions known as GM1 disease. GLB1 deficiency is characterized by the buildup of GM1 ganglioside that occurs in the brain and other organs.

The compositions provided herein, e.g., an HIR Ab-GLB1 fusion antibody, may be administered as part of a combination therapy. The combination therapy involves the administration of a composition of the present embodiments in combination with another therapy for treatment or relief of symptoms typically found in a patient suffering from a GLB1 deficiency. If the composition of the present embodiments is used in combination with another CNS disorder method or composition, any combination of the composition of the present embodiments and the additional method or composition may be used. Thus, for example, if use of a composition of the present embodiments is in combination with another CNS disorder treatment agent, the two may be administered simultaneously, consecutively, in overlapping durations, in similar, the same, or different frequencies, etc. In some cases a composition will be used that contains a composition of the present embodiments in combination with one or more other CNS disorder treatment agents.

In some embodiments, the composition, e.g., an HIR Ab-GLB1 fusion antibody is co-administered to the patient with another medication, either within the same formulation or as a separate composition. For example, the fusion antibody provided herein may be formulated with another fusion protein that is also designed to deliver across the human blood-brain barrier a recombinant protein other than GLB1. Further, the fusion antibody may be formulated in combination with other large or small molecules.

### EXAMPLES

The following specific examples are to be construed as merely illustrative, and not limitative of the remainder of the disclosure in any way whatsoever. Without further elaboration, it is believed that one skilled in the art may, based on the description herein, utilize the present embodiments to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1. Expression and Functional Analysis of HIR Ab-GUSB Fusion Protein

The lysosomal enzyme mutated in MPS-VII, also called Sly syndrome, is β-glucuronidase (GUSB). MPS-VII results in the accumulation in the brain of glycosoaminoglycans, which form lysosomal inclusion bodies. Enzyme replacement therapy (ERT) of MPS-VII would not likely be effective for treatment of the brain because the GUSB enzyme does not cross the BBB. In an effort to re-engineer human GUSB to cross the BBB, a HIR Ab-GUSB fusion protein project was initiated.

Human GUSB cDNA corresponding to amino acids Met₁-Thr₆₅₁ of the human GUSB protein (NP_000172), including the 22 amino acid signal peptide, and the 18 amino acid carboxyl terminal propeptide, was cloned by reverse transcription (RT) polymerase chain reaction (PCR) and custom oligodexoynucleotides (ODNs). PCR products were resolved in 1% agarose gel electrophoresis, and the expected major single band of ˜2.0 kb corresponding to the human GUSB cDNA was isolated. The cloned human GUSB was inserted into a eukaryotic expression plasmid, and this GUSB expression plasmid was designated pCD-GUSB. The entire expression cassette of the plasmid was confirmed by bi-directional DNA sequencing. Transfection of COS cells in a 6-well format with the pCD-GSUB resulted in high GUSB enzyme activity in the conditioned medium at 7 days (Table 1, Experiment A), which validated the successful engineering of a functional human GUSB cDNA. The GUSB enzyme activity was determined with a fluorometric assay using 4-methylumbelliferyl beta-L-glucuronide (MUGlcU), which is commercially available. This substrate is hydrolyzed to 4-methylumbelliferone (4-MU) by GUSB, and the 4-MU is detected fluorometrically with a fluorometer using an emission wavelength of 450 nm and an excitation wavelength of 365 nm. A standard curve was constructed with known amounts of 4-MU. The assay was performed at 37 C with 60 min incubations at pH=4.8, and was terminated by the addition of glycine-carbonate buffer (pH=10.5).

A new pCD-HC-GUSB plasmid expression plasmid was engineered, which expresses the fusion protein wherein the carboxyl terminus of the heavy chain (HC) of the HIR Ab is fused to the amino terminus of human GUSB, minus the 22 amino acid GUSB signal peptide, and minus the 18 amino acid carboxyl terminal GUSB propeptide. The GUSB cDNA was cloned by PCR using the pCD-GUSB as template. The forward PCR primer introduces “CA” nucleotides to maintain the open reading frame and to introduce a Ser-Ser linker between the carboxyl terminus of the CH3 region of the HIR Ab HC and the amino terminus of the GUSB minus the 22 amino acid signal peptide of the enzyme. The GUSB reverse PCR primer introduces a stop codon, “TGA,” immediately after the terminal Thr of the mature human GUSB protein. DNA sequencing of the expression cassette of the pCD-HC-GUSB encompassed 4,321 nucleotides (nt), including a 714 nt cytomegalovirus (CMV) promoter, a 9 nt Kozak site (GCCGCCACC), a 3,228 nt HC-GUSB fusion protein open reading frame, and a 370 nt bovine growth hormone (BGH) transcription termination sequence. The plasmid encoded for a 1,075 amino acid protein, comprised of a 19 amino acid IgG signal peptide, the 443 amino acid HIR Ab HC, a 2 amino acid linker (Ser-Ser), and the 611 amino acid human GUSB minus the enzyme signal peptide and carboxyl terminal propeptide. The GUSB sequence was 100% identical to Leu²³-Thr⁶³³ of human GUSB (NP_000172). The predicted molecular weight of the heavy chain fusion protein, minus glycosylation, is 119,306 Da, with a predicted isoelectric point (pI) of 7.83.

COS cells were plated in 6-well cluster dishes, and were dual transfected with pCD-LC and pCD-HC-GUSB, where pCD-LC is the expression plasmid encoding the light chain (LC) of the chimeric HIR Ab. Transfection was performed using Lipofectamine 2000, with a ratio of 1:2.5, ug DNA:uL Lipofectamine 2000, and conditioned serum free medium was collected at 3 and 7 days. However, there was no specific increase in GUSB enzyme activity following dual transfection of COS cells with the pCD-HC-GUSB and pCD-LC expression plasmids (Table 1, Experiment B). However, the low GUSB activity in the medium could be attributed to the low secretion of the HIR Ab-GUSB fusion protein, as the medium IgG was only 23±2 ng/mL, as determined by a human IgG-specific ELISA. Therefore, COS cell transfection was scaled up to 10×T500 plates, and the HIRMAb-GUSB fusion protein was purified by protein A affinity chromatography. IgG Western blotting demonstrated the expected increase in size of the fusion protein heavy chain. However, the GUSB enzyme activity of the HIRMAb-GUSB fusion protein was low at 6.1±0.1 nmol/hr/ug protein. In contrast, the specific activity of human recombinant GUSB is 2,000 nmol/hr/ug protein [Sands et al (1994) Enzyme replacement therapy for murine mucopolysaccharidosis type VII. J Clin Invest 93, 2324-2331]. These results demonstrated the GUSB enzyme activity of the HIR Ab-GUSB fusion protein was >95% lost following fusion of the GUSB to the carboxyl terminus of the HC of the HIR Ab. The affinity of HIR Ab-GUSB fusion protein binding to the extracellular domain (ECD) of the HIR was examined with an ELISA. CHO cells permanently transfected with the HIR ECD were grown in serum free media (SFM), and the HIR ECD was purified with a wheat germ agglutinin affinity column. The HIR ECD was plated on 96-well dishes and the binding of the HIR Ab, and the HIR Ab-GUSB fusion protein to the HIR ECD was detected with a biotinylated goat anti-human IgG (H+L) secondary antibody, followed by avidin and biotinylated peroxidase. The concentration of protein that gave 50% maximal binding, ED₅₀, was determined with a non-linear regression analysis. The HIR receptor assay showed there was no decrease in affinity for the HIR following fusion of the 611 amino acid GUSB to the carboxyl terminus of the HIRMAb heavy chain. The ED50 of the HIR Ab binding to the HIR ECD was 0.77±0.10 nM and the ED50 of binding of the HIR Ab-GUSB fusion protein was 0.81±0.04 nM.

In summary, fusion of the GUSB to the carboxyl terminus of the HIR Ab HC resulted in no loss in affinity of binding of the fusion protein to the HIR. However, the GUSB enzyme activity of the fusion protein was decreased by >95%.

In an effort to successfully produce a fusion protein of the HIR Ab and GUSB, a new approach was undertaken, in which the carboxyl terminus of the mature human GUSB, including the GUSB signal peptide, was fused to the amino terminus of the HC of the HIR Ab. This fusion protein was designated GUSB-HIR Ab. The first step was to engineer a new expression plasmid encoding this new fusion protein, and this plasmid was designated pCD-GUSB-HC. The pCD-GUSB-HC plasmid expresses the fusion protein wherein the amino terminus of the heavy chain (HC) of the HIRMAb, minus its 19 amino acid signal peptide, is fused to the carboxyl terminus of human GUSB, including the 22 amino acid GUSB signal peptide, but minus the 18 amino acid carboxyl terminal GUSB propeptide. The pCD-GUSB vector was used as template for PCR amplification of the GUSB cDNA expressing a GUSB protein that contained the 22 amino acid GUSB signal peptide, but lacking the 18 amino acid propeptide at the GUSB carboxyl terminus. The GUSB 18 amino acid carboxyl terminal propeptide in pCD-GUSB was deleted by site-directed mutagenesis (SDM). The latter created an AfeI site on the 3′-flanking region of the Thr⁶³³ residue of GUSB, and it was designated pCD-GUSB-AfeI. The carboxyl terminal propeptide was then deleted with AfeI and HindIII (located on the 3′-non coding region of GUSB). The HIRMAb HC open reading frame, minus the 19 amino acid IgG signal peptide and including the HIRMAb HC stop codon, was generated by PCR using the HIRMAb HC cDNA as template. The PCR generated HIRMAb HC cDNA was inserted at the AfeI-HindIII sites of pCD-GUSB-AfeI to form the pCD-GUSB-HC. A Ser-Ser linker between the carboxyl terminus of GUSB and amino terminus of the HIRMAb HC was introduced within the AfeI site by the PCR primer used for the cloning of the HIRMAb HC cDNA. DNA sequencing of the pCD-GUSB-HC expression cassette showed the plasmid expressed 1,078 amino acid protein, comprised of a 22 amino acid GUSB signal peptide, the 611 amino acid GUSB, a 2 amino acid linker (Ser-Ser), and the 443 amino acid HIRMAb HC. The GUSB sequence was 100% identical to Met¹-Thr⁶³³ of human GUSB (NP_000172).

Dual transfection of COS cells in a 6-well format with the pCD-LC and pCD-GUSB-HC expression plasmids resulted in higher GUSB enzyme activity in the conditioned medium at 7 days, as compared to dual transfection with the pCD-LC and pCD-HC-GUSB plasmids (Table 1, Experiment C). However, the GUSB-HIRMAb fusion protein was also secreted poorly by the COS cells, as the medium human IgG concentration in the 7 day conditioned medium was only 13±2 ng/mL, as determined by ELISA. COS cell transfection was scaled up to 10×T500 plates, and the GUSB-HIRMAb fusion protein was purified by protein A affinity chromatography. SDS-PAGE demonstrated the expected increase in size of the fusion protein heavy chain. The GUSB enzyme activity of the purified GUSB-HIRMAb fusion protein was high at 226±8 nmol/hr/ug protein, which is 37-fold higher than the specific GUSB enzyme activity of the HIRMAb-GUSB fusion protein. However, the HIR receptor assay showed there was a marked decrease in affinity for the HIR following fusion of the GUSB to the amino terminus of the HIRMAb heavy chain, which resulted in a 95% reduction in receptor binding affinity. The ED50 of the HIR Ab binding to the HIR ECD was 0.25±0.03 nM and the ED50 of binding of the HIR Ab-GUSB fusion protein was 4.8±0.4 nM.

In summary, fusion of the GUSB to the amino terminus of the HIR Ab HC resulted in retention of GUSB enzyme activity of the fusion protein, but caused a 95% reduction in binding of the GUSB-HIR Ab fusion protein to the HIR. In contrast, fusion of the GUSB to the carboxyl terminus of the HIR Ab HC resulted in no loss in affinity of binding of the HIR Ab-GUSB fusion protein to the HIR. However, the GUSB enzyme activity of this fusion protein was decreased by >95%. These findings may illustrate the unpredictable nature of the art of fusion of lysosomal enzymes to IgG molecules in such a way that bi-functionality of the IgG-enzyme fusion protein is retained, e.g., high affinity binding of the IgG part to the cognate antigen, as well as high enzyme activity.

TABLE 1
GUSB enzyme activity in COS cells following transfection
[Mean ± SE (n = 3 dishes per point)]

| | | Medium GUSB activity |
|---|---|---|
| Experiment | Treatment | (nmol/hour/mL) |
| A | Lipofectamine 2000 | 65 ± 1 |
| | pCD-GUSB | 6892 ± 631 |
| B | Lipofectamine 2000 | 76 ± 3 |
| | pCD-HC-GUSB, | 72 ± 3 |
| | pCD-LC | |
| C | Lipofectamine 2000 | 162 ± 7 |
| | pCD-HC-GUSB, | 155 ± 2 |
| | pCD-LC | |
| | pCD-GUSB-HC, | 1119 ± 54 |
| | pCD-LC | |

### Example 2. Expression and Functional Analysis of HIR Ab-GCR Fusion Protein

The lysosomal enzyme, mutated in Gaucher's disease (GD) is β-glucocerebrosidase (GCR). Neuronopathic forms of GD affect the CNS, and this results in accumulation of lysosomal inclusion bodies in brain cells, owing to the absence of GCR enzyme activity in the brain. Enzyme replacement therapy (ERT) of GD is not an effective for treatment of the brain because the GCR enzyme does not cross the BBB. In an effort to re-engineer human GCR to cross the BBB, a HIR Ab-GCR fusion protein project was engineered, expressed, and tested for enzyme activity. The human GCR cDNA corresponding to amino acids Ala⁴⁰-Gln⁵³⁶ of the human GCR protein (NP_000148), minus the 39 amino acid signal peptide, was custom synthesized by a commercial DNA production company. The GCR cDNA was comprised of 1522 nucleotides (nt), which included the GCR open reading frame, minus the signal peptide through the TGA stop codon. On the 5′-end, a StuI restriction endonuclease (RE) sequence was added, and on the 3′-end, a 14 nt fragment from the 3′-untranslated region of the GCR mRNA was followed by a HindIII RE site. Internal HindIII and StuI sites within the GCR gene were mutated without change of amino acid sequence. The GCR gene was released from the pUC plasmid provided by the vendor with StuI and HindIII, and was inserted at HpaI and HindIII sites of a eukaryotic expression plasmid encoding the HIR Ab heavy chain, and this expression plasmid was designated, pCD-HC-GCR. This expression plasmid expresses the fusion protein wherein the carboxyl terminus of the heavy chain (HC) of the HIR Ab is fused to the amino terminus of human GCR, minus the 39 amino acid GCR signal peptide, with a 3 amino acid linker (Ser-Ser-Ser) between the HIR Ab HC and the GCR. DNA sequencing confirmed the identity of the pCD-HC-GCR expression cassette. The expression cassette was comprised of 5,390 nt, which included a 2134 nt CMV promoter sequence, a 2,889 nt expression cassette, and a 367 BGH polyA sequence. The plasmid encoded for a 963 amino acid protein, which was comprised of a 19 amino acid IgG signal peptide, the 443 amino acid HIRMAb HC, a 3 amino acid linker (Ser-Ser-Ser), and the 497 amino acid human GCR minus the enzyme signal peptide. The GCR sequence was 100% identical to Als⁴⁰-Gln⁵³⁶ of human GCR (NP_000148). The predicted molecular weight of the heavy chain fusion protein, minus glycosylation, is 104,440 Da, with a predicted isoelectric point (pI) of 8.42. The HIR Ab-GCR fusion protein was expressed in transiently transfected COS cells. COS cells were plated in 6-well cluster dishes, and were dual transfected with pCD-LC and pCD-HC-GCR, where pCD-LC is the expression plasmid encoding the light chain (LC) of the chimeric HIR Ab. Transfection was performed using Lipofectamine 2000, with a ratio of 1:2.5, ug DNA:uL Lipofectamine 2000, and conditioned serum free medium was collected at 3 and 7 days. Fusion protein secretion into the serum free medium (SFM) was monitored by human IgG ELISA. The conditioned medium was clarified by depth filtration, and the HIR Ab-GCR fusion protein was purified by protein A affinity chromatography. The purity of the fusion protein was confirmed by reducing SDS-PAGE, and the identity of the fusion protein was confirmed by Western blotting using primary antibodies against either human IgG or human GCR. The IgG and GCR antibodies both reacted with the 130 kDa heavy chain of the HIR Ab-GCR fusion protein. The GCR enzyme activity of the fusion protein was measured with a fluorometric enzyme assay using 4-methylbumbelliferyl beta-D glucopyranoside (4-MUG) as the enzyme substrate as described previously for enzyme assay of recombinant GCR (J.B. Novo, et al, Generation of a Chinese hamster ovary cell line producing recombinant human glucocerebrosidase, J. Biomed. Biotechnol., Article ID 875383, 1-10, 2012). The GCR enzyme assay was performed with a final concentration of 4-MUG of 5 mM in citrate/phosphate buffer/pH=5.5 with 0.25% Triton X-100, and 0.25% sodium taurocholate, and the incubation was performed at 37 C for 60 minutes. Enzyme activity was stopped by the addition of 0.1 M glycine/0.1 M NaOH. The GCR enzyme converts the 4-MUG substrate to the product, 4-methlyumbelliferone (4-MU). An assay standard curve was constructed with 4-MU (0.03 to 3 nmol/tube). Enzyme activity was reported as units/mg protein, where 1 unit=1 umol/min. The enzyme activity of recombinant human GCR is 40 units/mg (Novo et al, 2012). However, the GCR enzyme activity of the HIR Ab-GCR fusion protein was only 0.07 units/mg, which is 99% reduced compared to the specific activity of recombinant GCR. This work showed that fusion of GCR to the C-terminus of the heavy chain of the HIR Ab with a short 3 amino acid linker resulted in a near complete loss of GCR enzyme activity.

The potential rescue of the GCR enzyme activity in the HIR Ab-GCR fusion protein was investigated further with the insertion of 3 different extended linkers between the CH3 domain of the HIR Ab HC and the GCR. The 3 extended linkers were comprised of 23, 31 or 58 amino acids in length, and these expression plasmids were designated, pCD-HC-GCR-L, pCD-HC-GCR-LL and pCD-HC-GCR-L4, respectively. In the pCD-HC-GCR-L, the linker corresponds to the 23 amino acids which comprise the sequence of the human IgG3 hinge region, and is derived from the 12 amino acids of the upper hinge region, followed by 5 amino acids of the first part of the core hinge region, and is flanked by a Ser-Ser-Ser sequence on the amino terminus and a Ser-Ser-Ser sequence on the carboxyl terminus. The 2 cysteine residues of the first part of the core hinge region are mutated to serine residues, so as to eliminate disulfide bonding. The sequence of the 23-amino acid linker is SSSELKTPLGDTTHTSPRSPSSS. In the pCD-HC-GCR-LL, the 31-amino acid linker corresponds to the 25 amino acids which comprise the sequence of the human IgG3 hinge region, and is derived from the 12 amino acids of the upper hinge region, followed by 5 amino acids of the first part of the core hinge region, followed by 8 amino acids of the lower hinge region, and is flanked by a Ser-Ser-Ser sequence on the amino terminus and a Ser-Ser-Ser sequence on the carboxyl terminus. The 2 cysteine residues of the first part of the core hinge region are mutated to serine residues, so as to eliminate disulfide bonding. The sequence of the 31-amino acid linker is SSSELKTPLGDTTHTSPRSPAPEFLGGPSSS. In the pCD-HC-GCR-L4, the 58-amino acid linker corresponds to 2 repeats of the 25 amino acids which comprise the sequence of the human IgG3 hinge region, and is derived from the 12 amino acids of the upper hinge region, followed by 5 amino acids of the first part of the core hinge region, followed by 8 amino acids of the lower hinge region, separate by a Ser-Ser residues and flanked by a Ser-Ser-Ser sequence on the amino terminus and a Ser-Ser-Ser sequence on the carboxyl terminus. The 2 cysteine residues of the first part of the core hinge region of either repeat are mutated to serine residues, so as to eliminate disulfide bonding. The sequence of the 58-amino acid linker is SSSELKTPLGDTTHTSPRSPAPEFLGGPSSELKTPLGDTTHTSPRSPAPEFLGGPSSS. The 5′-end of the GCR cDNA was linked to the cDNA encoding the HC of the HIR Ab via the 23, 31 or 58 amino acid linker. These expression plasmids express fusion proteins wherein the carboxyl terminus of the heavy chain (HC) of the HIR Ab is fused to the amino terminus of human GCR, minus the 39 amino acid GCR signal peptide, with either a 23, 31 or 58 amino acid linker between the C-terminus of the HIR Ab HC and the N-terminus of the mature GCR, respectively. DNA sequencing confirmed the identity of the 3 pCD-HC-GCR expression cassettes. The plasmids encoded for proteins of 983, 991 and 1,018 amino acids, respectively, which were comprised of a 19 amino acid IgG signal peptide, the 443 amino acid HIRMAb HC, 23, 31 or 58 amino acid linker, and the 497 amino acid human GCR minus the enzyme signal peptide. The GCR sequence was 100% identical to Als⁴⁰-Gln⁵³⁶ of human GCR (NP_000148). The HIR Ab-GCR fusion proteins with the extended linkers were expressed in transiently transfected COS cells. COS cells were dual transfected with pCD-LC and pCD-HC-GCR-L, pCD-HC-GCR-LL or pCD-HC-GCR-L4, where pCD-LC is the expression plasmid encoding the light chain (LC) of the chimeric HIR Ab. Transfection was performed using Lipofectamine 2000, with a ratio of 1:2.5, ug DNA:uL Lipofectamine 2000, and conditioned serum free medium was collected at 3 and 7 days. Fusion protein secretion into the serum free medium (SFM) was monitored by human IgG ELISA. The conditioned medium was clarified by depth filtration, and the HIR Ab-GCR fusion protein was purified by protein A affinity chromatography. The purity of the fusion protein was confirmed by reducing SDS-PAGE, and the identity of the fusion protein was confirmed by Western blotting using primary antibodies against either human IgG or human GCR. The GCR enzyme activity of the fusion proteins with the extended linkers was measured with a fluorometric enzyme assay using 4-methylbumbelliferyl beta-D glucopyranoside (4-MUG) as the enzyme substrate as described above, and previously for enzyme assay of recombinant GCR (J.B. Novo, et al, Generation of a Chinese hamster ovary cell line producing recombinant human glucocerebrosidase, J. Biomed. Biotechnol., Article ID 875383, 1-10, 2012). The GCR enzyme assay was performed with a final concentration of 4-MUG of 5 mM in citrate/phosphate buffer/pH=5.5 with 0.25% Triton X-100, and 0.25% sodium taurocholate, and the incubation was performed at 37 C for 60 minutes. Enzyme activity was stopped by the addition of 0.1 M glycine/0.1 M NaOH. The GCR enzyme converts the 4-MUG substrate to the product, 4-methlyumbelliferone (4-MU). An assay standard curve was constructed with 4-MU (0.03 to 3 nmol/tube). Enzyme activity was reported as units/mg protein, where 1 unit=1 umol/min. The GCR enzyme activity of the HIR Ab-GCR-L, -LL or -L4 fusion proteins was only <5% of the specific activity of recombinant GCR. These results show that fusion of the GCR to the C-terminus of the heavy chain (HC) IgG, even with long linkers ranging from 23 to 58 amino acids in length, results in a near complete loss of GCR enzyme activity.

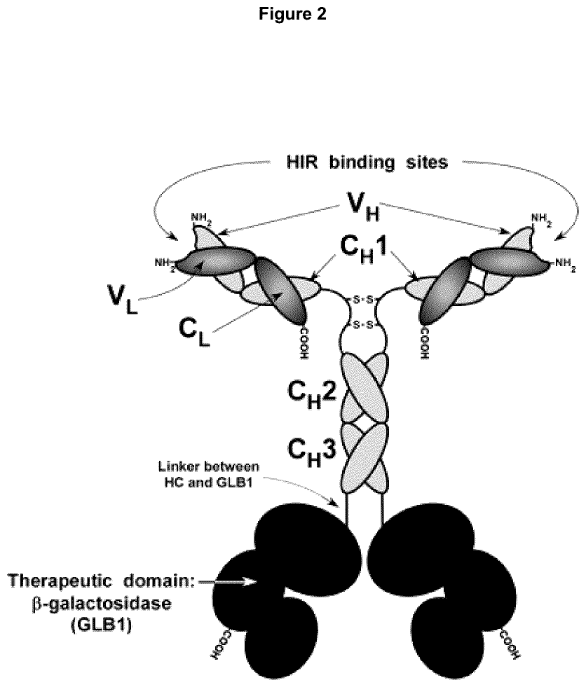

In a further effort to rescue the GCR enzyme activity in HIR Ab-GCR fusion protein, another construct was engineered, wherein the human GCR cDNA is fused to the C-terminus of the light chain (LC) of the HIR Ab via a 31 amino acid linker. The human GCR protein (NP_000148), minus the 39 amino acid signal peptide was custom synthesized by a commercial DNA production company. The GCR cDNA was comprised of 1522 nucleotides (nt), which included the GCR open reading frame, minus the signal peptide through the TGA stop codon. The 5′-end of the GCR cDNA was linked to the 702 nt cDNA encoding the light chain (LC) of the HIR Ab via a 31 amino acid linker. This linker corresponds to the 25 amino acids which comprise the sequence of the human IgG3 hinge region, and is derived from the 12 amino acids of the upper hinge region, followed by 5 amino acids of the first part of the core hinge region, followed by 8 amino acids of the lower hinge region, and is flanked by a Ser-Ser-Ser sequence on the amino terminus and a Ser-Ser-Ser sequence on the carboxyl terminus. The 2 cysteine residues of the first part of the core hinge region are mutated to serine residues, so as to eliminate disulfide bonding. The sequence of the 31-amino acid linker is SSSELKTPLGDTTHTSPRSPAPEFLGGPSSS. A 18 nt fragment from the 3′-untranslated region of the expression vector was added on the on the 3′-end followed by a PmeI RE site. The 5′-end of the fusion protein cDNA contains an EcoRI site followed by 5 nt of the 5′-untranslated region of the expression vector followed by a complete Kozak site (GCCGCCACC). The artificial gene coding for the HIR Ab-LC-GCR was comprised of 2,335 base pairs and it was custom synthesized by a commercial DNA production company. The HIR Ab LC-GCR gene was released from the pUC plasmid provided by the vendor with EcoRI and PmeI, and was inserted at same RE sites of a eukaryotic expression vector flanking by the CMV promoter and the BGH polyA region, respectively, to form an expression plasmid designated pHIR Ab LC-GCR. This expression plasmid expresses the fusion protein wherein the carboxyl terminus of the LC of the HIR Ab is fused to the amino terminus of human GCR, minus the 39 amino acid GCR signal peptide, with a 31 amino acid linker (SSSELKTPLGDTTHTSPRSPAPEFLGGPSSS) between the HIR Ab LC and the GCR. DNA sequencing confirmed the identity of the pHIR-LC-GCR expression cassette. The expression cassette was comprised of 4,444 nt, which included a 1,855 nt CMV promoter sequence, a 9 Kozak site, a 2,298 nt fusion protein cDNA, and a 291 BGH polyA sequence. The plasmid encoded for a 762 amino acid LC-GCR fusion protein, which was comprised of a 20 amino acid IgG signal peptide, the 214 amino acid HIR Ab LC, a 31 amino acid linker, and the 497 amino acid human GCR minus the enzyme signal peptide. The GCR sequence was 100% identical to Als⁴⁰-Gln⁵³⁶ of human GCR (NP_000148). The HIR Ab LC-GCR fusion protein was expressed in transiently transfected COS cells. COS cells were plated and were dual transfected with expression plasmids encoding the HIR Ab LC-GCR and HIR Ab HC, where the latter is the heavy chain (HC) of the chimeric HIR Ab (FIG. 2). The pHIR Ab-HC encodes for the 443 amino acid sequence of the HIR Ab-HC protein. The 2,328 nt sequence encoding the HIR Ab-HC is comprised of a 9 nt Kozak sequence (GCCGCCACC), followed by a 2,316 nt sequence encoding the open reading frame followed by a TAA stop codon. Transfection was performed using Lipofectamine 2000, with a ratio of 1:2.5, ug DNA:uL Lipofectamine 2000, and conditioned serum free medium was collected at 3 and 7 days. Fusion protein secretion into the serum free medium (SFM) was monitored by human IgG ELISA. The conditioned medium was clarified by ultrafiltration filtration, and the HIR Ab-GCR fusion protein was purified by protein A affinity chromatography. The purity of the fusion protein was confirmed by reducing SDS-PAGE. The identity of the HIR Ab-GCR fusion protein was confirmed by Western blotting using antibodies against human IgG and human GCR. The GCR enzyme activity of the HIR Ab-LC-GCR fusion protein was measured with a fluorometric enzyme assay using 4-methylbumbelliferyl beta-D glucopyranoside (4-MUG), as described above. The GCR enzyme activity of the HIR Ab-LC-GCR fusion protein was only <5% of the specific activity of recombinant GCR. These combined results show that fusion of a lysosomal enzyme, GCR, to the C-terminus of either the HC or the LC of an IgG with linkers of different lengths, resulted in an IgG-enzyme fusion protein that was nearly devoid of enzyme activity.

In a continued effort to retain the GCR enzyme activity in a HIR Ab-GCR fusion protein, another construct was re-engineered, wherein the human GCR, including its signal peptide, is fused to the N-terminus of the heavy chain (HC) of the HIR Ab via a 56 amino acid linker between the C-terminus of the GCR and the N-terminus of the antibody HC. A custom gene was synthesized, which encoded the sequence of human GCR protein including the 39 amino acid signal peptide of GCR, as provided in NP_000148, is followed by a 56 amino acid linker, described below, followed by the 443 amino acid HIR Ab heavy chain with the heavy chain signal peptide. This gene contains 20 nt of the 5′-flanking region including an EcoRI site part of the promoter region and the full length Kozak site. On the 3′-flanking region, the gene contains 291 nt corresponding to the BGH polyA site followed by 30 nt of untranslated region including a NheI site. The 3,449 nt gene was custom synthesized by a commercial vendor. The 56-amino acid linker corresponds to 2 repeats of the 25 amino acids which comprise the sequence of the human IgG3 hinge region, and is derived from the 12 amino acids of the upper hinge region, followed by 5 amino acids of the first part of the core hinge region, followed by 8 amino acids of the lower hinge region, separate by a Ser-Ser residues and flanked by a Ser residue on the amino terminus and a Ser-Ser-Ser sequence on the carboxyl terminus. The 2 cysteine residues of the first part of the core hinge region of either repeat are mutated to serine residues, so as to eliminate disulfide bonding. The sequence of the 56-amino acid linker is SELKTPLGDTTHTSPRSPAPEFLGGPSSELKTPLGDTTHTSPRSPAPEFLGGPSSS. The GCR-HIR Ab HC gene was released from the pUC plasmid provided by the vendor with EcoRI and NheI, and was inserted at same RE sites of a eukaryotic expression vector flanking by the CMV promoter to form an expression plasmid designated pGCR-HIR-Ab-HC. DNA sequencing confirmed the identity of the pGCR-HIR-Ab-HC expression cassette. The expression cassette was comprised of 5,263 nt, which included a 1,855 nt CMV promoter sequence, a 9 nt Kozak site, a 3,108 nt fusion protein cDNA, and a 291 BGH polyA sequence. The plasmid encoded for a 1,035 amino acid GCR-HIR Ab HC fusion protein, which was comprised of a 39 amino acid GCR signal peptide, the 497 amino acid GCR, a 56 amino acid linker, and the 443 amino acid HIR Ab HC minus the IgG signal peptide. The GCR sequence was 100% identical to Met¹-Gln⁵³⁶ of human GCR (NP_000148). The GCR-HIR Ab HC fusion protein was expressed in transiently transfected COS cells. COS cells were plated and were dual transfected with expression plasmids encoding the GCR-HIR Ab HC and HIR Ab LC, where the latter is the light chain (LC) of the chimeric HIR Ab (FIG. 2). The pHIR Ab-LC encodes for the 234 amino acid sequence of the HIR Ab-LC protein. The 714 nt sequence encoding the HIR Ab-LC is comprised of a 9 nt Kozak sequence (GCCGCCACC), followed by a 705 nt sequence encoding the open reading frame followed by a TAG stop codon. Transfection was performed using Lipofectamine 2000, with a ratio of 1:2.5, ug DNA:uL Lipofectamine 2000, and conditioned serum free medium was collected at 3 and 7 days. Fusion protein secretion into the serum free medium (SFM) was monitored by human IgG ELISA. The conditioned medium was clarified by ultrafiltration filtration, and the HIR Ab-GCR fusion protein was purified by protein A affinity chromatography. The purity of the fusion protein was confirmed by reducing SDS-PAGE. The identity of the HIR Ab-GCR fusion protein was confirmed by Western blotting using antibodies against human IgG and human GCR. The GCR enzyme activity of the HIR Ab-LC-GCR fusion protein was measured with a fluorometric enzyme assay using 4-methylbumbelliferyl beta-D glucopyranoside (4-MUG), as described above. The GCR enzyme activity of the HIR Ab-LC-GCR fusion protein was only <7% of the specific activity of recombinant GCR. These combined results on the engineering of an enzymatically active IgG-GCR fusion protein describe the engineering of 5 different constructs, wherein the GCR was fused to either the C-terminus or the N-terminus of either the HC or the LC of the IgG, and with a variety of linkers ranging from 3, 23, 31, 56, or 58 amino acids. In all cases, the IgG-enzyme fusion protein that was nearly devoid of enzyme activity.

### Example 3. Expression and Functional Analysis of HIR Ab-GALC Fusion Protein

The lysosomal enzyme, mutated in Krabbe disease (KD) is galactocerebrosidase (GALC). KD is a rare neurodegenerative disorder that affects the myelin sheath of the nervous system involving dysfunctional metabolism of sphingolipids. Enzyme replacement therapy (ERT) of KD is not an effective treatment of the brain because the GALC enzyme does not cross the BBB. In an effort to re-engineer human GALC to cross the BBB, a HIR Ab-GALC fusion protein project was engineered, expressed, and tested for enzyme activity. The human GALC cDNA corresponds to amino acids Tyr₄₃-Arg₆₈₅ of the human GALC protein (NM_000153), minus the 42 amino acid signal peptide. The GALC cDNA was comprised of 1,932 nucleotides (nt), which included the GALC open reading frame, minus the signal peptide through the TGA stop codon. The 5′-end of the GALC cDNA was linked to the 702 nt cDNA encoding the light chain (LC) of the HIR Ab via a 31 amino acid linker. This linker corresponds to the 25 amino acids which comprise the sequence of the human IgG3 hinge region, and is derived from the 12 amino acids of the upper hinge region, followed by 5 amino acids of the first part of the core hinge region, followed by 8 amino acids of the lower hinge region, and is flanked by a Ser-Ser-Ser sequence on the amino terminus and a Ser-Ser-Ser sequence on the carboxyl terminus. The 2 cysteine residues of the first part of the core hinge region are mutated to serine residues, so as to eliminate disulfide bonding. The sequence of the 31-amino acid linker is SSSELKTPLGDTTHTSPRSPAPEFLGGPSSS. A 18 nt fragment from the 3′-untranslated region of the expression vector was added on the on the 3′-end followed by a PmeI RE site. The 5′-end of the fusion protein cDNA contains an EcoRI site followed by 5 nt of the 5′-untranslated region of the expression vector followed by a complete Kozak site (GCCGCCACC). The artificial gene coding for the HIR Ab-LC-GALC was comprised of 2,776 base pairs and it was custom synthesized by a commercial DNA production company. The HIR Ab LC-GALC gene was released from the pUC plasmid provided by the vendor with EcoRI and PmeI, and was inserted at same RE sites of a eukaryotic expression vector flanking by the CMV promoter and the BGH polyA region, respectively, to form an expression plasmid designated pHIR Ab LC-GALC. This expression plasmid expresses the fusion protein wherein the carboxyl terminus of the LC of the HIR Ab is fused to the amino terminus of human GALC, minus the 42 amino acid GALC signal peptide, with a 31 amino acid linker (SSSELKTPLGDTTHTSPRSPAPEFLGGPSSS) between the HIR Ab LC and the GALC. DNA sequencing confirmed the identity of the pHIR-LC-GALC expression cassette. The expression cassette was comprised of 4,885 nt, which included a 1,855 nt CMV promoter sequence, a 2,736 nt fusion protein cDNA, and a 294 BGH polyA sequence. The plasmid encoded for a 908 amino acid LC-GALC fusion protein, which was comprised of a 20 amino acid IgG signal peptide, the 214 amino acid HIR Ab LC, a 31 amino acid linker, and the 643 amino acid human GALC minus the enzyme signal peptide. The GALC sequence was 100% identical to Tyr₄₃-Arg₆₈₅ of the human GALC protein (NM_000153). The predicted molecular weight of the light chain fusion protein, minus glycosylation, is 99,363 Da, with a predicted isoelectric point (pI) of 5.8. The HIR Ab-GALC fusion protein was expressed in transiently transfected COS cells. COS cells were plated and were dual transfected with expression plasmids encoding the HIR Ab LC-GALC and pHIR Ab HC, where the latter is the heavy chain (HC) of the chimeric HIR Ab (FIG. 2). The pHIR Ab-HC encodes for the 443 amino acid sequence of the HIR Ab-HC protein. The 2,328 nt sequence encoding the HIR Ab-HC is comprised of a 9 nt Kozak sequence (GCCGCCACC), followed by a 2,316 nt sequence encoding the open reading frame followed by a TAA stop codon. Transfection was performed using Lipofectamine 2000, with a ratio of 1:2.5, ug DNA:uL Lipofectamine 2000, and conditioned serum free medium was collected at 3 and 7 days. Fusion protein secretion into the serum free medium (SFM) was monitored by human IgG ELISA. The conditioned medium was clarified by ultrafiltration filtration, and the HIR Ab-GALC fusion protein was purified by protein A affinity chromatography. The purity of the fusion protein was confirmed by reducing SDS-PAGE, which showed the expected light chain-GALC fusion and the heavy chain of the fusion protein, which migrated at molecular weights of 115 and 55 kDa, respectively. The identity of the HIR Ab-GALC fusion protein was confirmed by Western blotting using antibodies against human IgG and human GALC. The GALC enzyme activity of the fusion protein was measured with a fluorometric enzyme assay using 4-methylumbelliferyl-beta-D-galactopyranoside (MUGP), as the enzyme substrate as described previously for enzyme assay of recombinant GALC (Meng et al., Proc Natl Acad Sci, 107:7886-91, 2010). The GALC enzyme assay was performed with a final concentration of MUGP of 0.5 mM in citrate/sodium chloride buffer/pH=4.5 with 0.25% Triton X-100, and the incubation was performed at 37 C for 20 minutes. Enzyme activity was stopped by the addition of 0.25 M glycine/0.15 M NaOH. The GALC enzyme converts the MUGP substrate to the product, 4-methlyumbelliferone (4-MU). An assay standard curve was constructed with 4-MU (0.01 to 3 nmol/tube). Enzyme activity was reported as units/mg protein, where 1 unit=1 nmol/min. The enzyme activity of the HIR Ab-GALC fusion protein was compared in the same enzyme assay with commercially available recombinant human GALC. The human recombinant GALC had high enzyme activity of 1845 units/mg. However, the GALC enzyme activity of the HIR Ab-GALC fusion protein was only 13.3 units/mg, which is 99% reduced compared to the specific activity of recombinant GALC.

In an effort to rescue the GALC enzyme activity in HIR Ab-GALC fusion protein, another construct was re-engineered, wherein the human GALC cDNA, including its signal peptide, is fused to the N-terminus of the light chain (LC) of the HIR Ab via a 31 amino acid linker. The cDNA encoded for the human GALC protein including the 42 amino acid signal peptide of the enzyme (NM_000153) followed by a 31 amino acid linker followed by the 214 amino acid sequence of the HIR-Ab LC without the LC signal peptide. This gene contains 20 nt of the 5′-flanking region including an EcoRI site part of the promoter region and the full length Kozak site. On the 3′-flanking region, the gene contains 29 nt corresponding to part of the BGH polyA site followed by 30 nt of untranslated region including a PmeI site. The 2,842 nt gene was custom synthesized by a commercial vendor. The 31-amino acid linker corresponds to the 25 amino acids which comprise the sequence of the human IgG3 hinge region, and is derived from the 12 amino acids of the upper hinge region, followed by 5 amino acids of the first part of the core hinge region, followed by 8 amino acids of the lower hinge region, and flanked by a Ser-Ser-Ser sequence on the amino terminus and a Ser-Ser-Ser sequence on the carboxyl terminus. The 2 cysteine residues of the first part of the core hinge region are mutated to serine residues, so as to eliminate disulfide bonding. The sequence of the 31-amino acid linker is SSSELKTPLGDTTHTSPRSPAPEFLGGPSSS. The GALC-HIR Ab LC gene was released from the pUC plasmid provided by the vendor with EcoRI and PmeI, and was inserted at same RE sites of a eukaryotic expression vector flanking by the CMV promoter to form an expression plasmid designated pGALC-HIR-Ab-LC. This expression plasmid expresses the fusion protein wherein the carboxyl terminus of the GALC is fused to the amino terminus of HIR Ab LC, minus the 20 amino acid HIR Ab LC signal peptide, with a 31 amino acid linker between the GALC and HIR Ab LC. DNA sequencing confirmed the identity of the pGALC-HIR-Ab-LC expression cassette. The expression cassette was comprised of 4,951 nt, which included a 1,855 nt CMV promoter sequence, a 9 nt Kozak site, a 27,93 nt fusion protein cDNA, and a 294 BGH polyA sequence. The plasmid encoded for a 930 amino acid GALC-HIR Ab LC fusion protein, which was comprised of a 42 amino acid GALC signal peptide, the 643 amino acid GALC, a 31 amino acid linker, and the 214 amino acid HIR Ab LC minus the IgG signal peptide. The GALC sequence was 100% identical to Met₁-Arg₆₈₅ of human GALC (NM_000153). The GALC-HIR Ab LC fusion protein was expressed in transiently transfected COS cells. COS cells were plated and were dual transfected with expression plasmids encoding the GALC-HIR Ab LC and HIR Ab HC, where the latter is the heavy chain (HC) of the chimeric HIR Ab (FIG. 2). The pHIR Ab-HC encodes for the 443 amino acid sequence of the HIR Ab-HC protein. The 2,328 nt sequence encoding the HIR Ab-HC is comprised of a 9 nt Kozak sequence (GCCGCCACC), followed by a 2,316 nt sequence encoding the open reading frame followed by a TAA stop codon. Transfection was performed using Lipofectamine 2000, with a ratio of 1:2.5, ug DNA:uL Lipofectamine 2000, and conditioned serum free medium was collected at 3 and 7 days. Fusion protein secretion into the serum free medium (SFM) was monitored by human IgG ELISA. The conditioned medium was clarified by ultrafiltration filtration, and the GALC-HIR Ab-LC fusion protein was purified by protein A affinity chromatography. The purity of the fusion protein was confirmed by reducing SDS-PAGE. The identity of the GALC-HIR Ab-LC fusion protein was confirmed by Western blotting using antibodies against human IgG and human GALC. The GALC enzyme activity of the GALC-HIR Ab-LC fusion protein was measured with a fluorometric enzyme assay using 4-methylumbelliferyl-beta-D-galactopyranoside (MUGP), as described above. The GALC enzyme activity of the GALC-HIR Ab-LC fusion protein was only <5% of the specific activity of recombinant GALC. These combined results show that fusion of a lysosomal enzyme, GALC, to the C-terminus or the N-terminus of the LC of an IgG resulted in an IgG-enzyme fusion protein that was nearly devoid of enzyme activity.

In the case of the GUSB, enzyme activity was lost following fusion to the C-terminus (CT) of the heavy chain (HC) of the HIR Ab. Conversely, enzyme activity was retained following fusion of GUSB to the amino terminus (NT) of the HC of the HIR Ab, but in this case, >95% binding activity of the antibody for the human insulin receptor (HIR) was lost. The loss of binding to the HIR may be because the CDR regions, which bind the target antigen, are located near the NT of the HC or light chain (LC). In the case of fusion of GCR to either the NT or the CT of either the HC or the LC of the HIR Ab, with linkers of variable length ranging from 3 amino acids to 58 amino acids, there was a loss of GCR enzyme activity of >95%. In the case of GALC, the enzyme was fused to either the CT or the NT of the LC of the antibody with 31 amino acid long linkers, but this resulted in a near complete loss of enzyme activity for either construct. In the present invention, we make the unexpected observation that GLB1 activity is retained following fusion of the GLB1 to the carboxyl terminus of the HIR Ab (FIG. 2).

### Example 4. Genetic Engineering of a HIR Ab Heavy Chain-GLB1 Fusion Protein Expression Plasmid DNA

The lysosomal enzyme mutated in GM1 is GLB1. Loss of GLB1 results in accumulation of GM1 ganglioside in the brain, and peripheral organs. Intravenous enzyme replacement therapy of GM1 is not effective for treatment of the brain because the GM1 enzyme does not cross the BBB, as demonstrated for the bacterial form of the enzyme (Zhang and Pardridge; Delivery of beta-galactosidase to mouse brain via the blood brain barrier transferrin receptor, J. Pharm. Exp. Ther., 313, 1075-1081). To enable BBB transfer of the enzyme, GLB1 was engineered as an IgG-GLB1 fusion protein, where the GLB1 was fused to the HIR Ab. The goal is to develop a bifunctional molecule capable of both crossing the BBB and exhibiting high GLB1 enzymatic activity. In one embodiment the amino terminus of the mature GLB1 is fused to the carboxyl terminus of each heavy chain of the HIR Ab (FIG. 2).

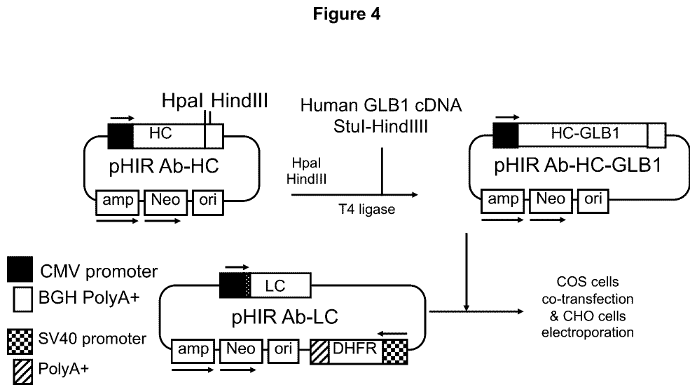

It was unclear whether the enzymatic activity of the GLB1 would be retained when it was fused to the HIR Ab. The experience with IgG-GUSB, IgG-GCR, and IgG-GALC fusion proteins described in Examples 1, 2, and 3 demonstrates that either the IgG part or the lysosomal enzyme part could lose biological activity following construction of the IgG-enzyme fusion protein. The human GLB1 cDNA corresponds to amino acids Leu-24 to Val-677 of the human GLB1 protein (accession #NP_000395), minus the 23 amino acid signal peptide. Initially, the HIR Ab-GLB1 fusion protein was engineered where the GLB1, without the enzyme signal peptide was fused to the C-terminus of the heavy chain (HC) of the HIR Ab with a short linker (SL) of 4-amino acids. However, as described below, this fusion protein had reduced GLB1 enzyme activity. Therefore, the engineering of the HIR Ab-GLB1 fusion protein was re-designed wherein the 4-amino acid linker between the C-terminus of the HC and the N-terminus of the GLB1 was replaced with a long linker (LL) of 31-amino acids. The GLB1 fusion protein with the short 4-amino acid linker is designated HIR Ab-SL-GLB1. The GLB1 fusion protein with the long 31-amino acid linker is designated HIR Ab-LL-GLB1. In the HIR Ab-SL-GLB1 fusion protein, the linker corresponds to the Ser-Ser-Ser-Ser sequence of amino acids 462-465 of SEQ ID NO:10 in (FIG. 9). In the HIR Ab-LL-GLB1 fusion protein, the 31-amino acid linker corresponds to amino acids 462-492 of SEQ ID NO:11 (FIG. 10). This 31-amino acid linker is comprised of 25 amino acids from the human IgG3 hinge region, and is derived from the 12 amino acids of the upper hinge region, followed by 5 amino acids of the first part of the core hinge region, followed by 8 amino acids of the lower hinge region, and is flanked by a Ser-Ser-Ser sequence on the amino terminus and a Ser-Ser-Ser sequence on the carboxyl terminus. The 2 cysteine residues of the first part of the core hinge region are mutated to serine residues, so as to eliminate disulfide bonding. The sequence of the 31-amino acid linker is SSSELKTPLGDTTHTSPRSPAPEFLGGPSSS. The human GLB1 cDNA corresponding to amino acids Leu₂₄ to Val₆₇₇ of the human GLB1 protein (accession #NP_000395), minus the 23 amino acid signal peptide, was custom synthesized by a commercial DNA production company. The GLB1 cDNA was comprised of 1,996 nucleotides (nt), which included the GLB1 open reading frame, minus the signal peptide through the TGA stop codon. On the 5′-end, a StuI restriction endonuclease (RE) sequence was added, followed by CA nt to maintain the open reading frame with CH3 and linker regions of the fusion protein. On the 3′-end, a 23 nt fragment was added corresponding to the 3′-untranslated region of the expression vector including a HindIII RE site. The GLB1 gene was released from the pUC plasmid provided by the vendor with StuI and HindIII, and was inserted at HpaI and HindIII sites of a eukaryotic expression plasmid encoding the HIR Ab heavy chain, and this expression plasmid was designated, pHIR Ab-HC-GLB1 (FIG. 4). The 5′-end of the GLB1 cDNA was linked to the cDNA encoding the HC of the HIR Ab via the 4 or the 31 amino acid linker. The terminal lysine residue at position 462 of the HIR Ab HC (SEQ ID NO: 7) was deleted in the fusion protein as this is a potential protease site. These expression plasmids express fusion proteins wherein the carboxyl terminus of the heavy chain (HC) of the HIR Ab is fused to the amino terminus of human GLB1, minus the 23 amino acid GLB1 signal peptide, with either a 4 or 31 amino acid linker between the C-terminus of the HIR Ab HC and the N-terminus of the mature GLB1, respectively. DNA sequencing confirmed the identity of the pHIR Ab-HC-GLB1 expression cassettes corresponding to the HIR Ab-SL-GLB1 and HIR Ab-LL-GLB1 fusion proteins, respectively. The expression cassettes were comprised of or 5,864 or 5,945 nt, for HIR Ab-SL-GLB1 (FIG. 9, SEQ ID NO:10) and HIR Ab-LL-GLB1 (FIG. 10, SEQ ID NO:11) fusion protein, respectively, which included 2,125 nt CMV promoter, 9 nt Kozak site, 3,360 or 3,441 nt open reading frame, and a 370 nt BGH polyA sequence. The plasmids encoded for proteins of 1,119 and 1,146 amino acids, respectively, which were comprised of a 19 amino acid IgG signal peptide, the 442 amino acid HIRMAb HC, 4 or 31 amino acid linker, and the 654 amino acid human GLB1 minus the enzyme signal peptide. The GLB1 sequence was 100% identical to amino acids Leu-24 to Val-677 of the human GLB1 protein (accession #NP_000395). The predicted molecular weight of the HC of the HIR Ab-SL-GLB1 and HIR Ab-LL-GLB1 fusion protein, respectively, minus glycosylation, is 122,398 Da and 125,160 Da, respectively, with a predicted isoelectric point (pI) of 6.79 and 6.71, respectively. The expression vector of pHIR Ab-LC also contains in tandem an expression cassette for the dihydrofolate reductase (DHFR) (FIG. 4), which is used to generate stable transfectants in CHO cells. The 187 amino acid sequence of the DHFR selection protein is given in SEQ ID NO:17. The 573 nt sequence encoding the DHFR is given in SEQ ID NO: 16, which is comprised of a 9 nt Kozak sequence (GCCGCCACC), followed by a 561 nt sequence encoding the open reading frame followed by a TAA stop codon.

The HIR Ab-SL-GLB1 and HIR Ab-LL-GLB1 fusion proteins were expressed in transiently transfected COS cells. COS cells were dual transfected with pCD-LC and expression plasmids for HIR Ab-SL-GLB1 or HIR Ab-LL-GLB1, where pCD-LC is the expression plasmid encoding the light chain (LC) of the chimeric HIR Ab (FIG. 4). The pHIR Ab-LC encodes for the 234 amino acid sequence of the HIR Ab-HC protein, and corresponds to amino acid sequence in SEQ ID NO:8. The 714 nt sequence encoding the HIR Ab-LC is given in SEQ ID NO:14, which is comprised of a 9 nt Kozak sequence (GCCGCCACC), followed by a 702 nt sequence encoding the open reading frame followed by a TAA stop codon. Transfection was performed using Lipofectamine 2000, with a ratio of 1:2.5, ug DNA:uL Lipofectamine 2000, and conditioned serum free medium was collected at 3 and 7 days. Fusion protein secretion into the serum free medium (SFM) was monitored by human IgG ELISA. The conditioned medium was clarified by ultrafiltration filtration, and the HIR Ab-GLB1 fusion protein was purified by protein A affinity chromatography.

### Example 5. Stable Transfection of Chinese Hamster Ovary Cells with Expression Vectors Encoding Both Heavy and Light Chains of the HIRMAb-GLB1 Fusion Protein

Chinese hamster ovary (CHO) cells were grown in serum free CHO utility medium, containing 1×HT supplement (hypoxanthine and thymidine). CHO cells (5×10⁶ viable cells) were co-electroporated with 2.5 μg PvuI-linearized pHIR Ab HC-GLB1 and 2.5 μg PvuI-linearized pHIR Ab-LC plasmid DNA for expression of HIR Ab-LL-GLB1 (FIG. 4). The cell-DNA suspension was incubated for 10 min on ice. Cells were square wave electroporated with a pulse of 25 msec and 160 volts. After electroporation (EP), cells were incubated for 10 min on ice. The cell suspension was transferred to 50 ml culture medium and plated at 125 μl per well in 4×96-well plates (10,000 cells per well). Following EP, the CHO cells were placed in the incubator at 37 C and 8% CO2. Owing to the presence of the neomycin resistance (neo) gene in the expression vector (FIG. 4), transfected cell lines were initially selected with G418. The pHIR Ab LC also expresses the gene for DHFR (FIG. 4), so the transfected cells were also selected with 20 nM methotrexate (MTX) and HT deficient medium. Once visible colonies were detected at about 21 days after EP, the conditioned medium was sampled for human IgG by ELISA. Wells with high human IgG signals in the ELISA were transferred from the 96-well plate to a 24-well plate with 1 mL of CHO-Utility serum free medium. The 24-well plates were returned to the incubator at 37 C and 8% CO2. The following week IgG ELISA was performed on the clones in the 24-well plates. This was repeated through the 6-well plates to T75 flasks and finally to 60 mL and 125 mL square plastic bottles on an orbital shaker. At this stage, the final MTX concentration was 80 nM, and the medium IgG concentration, which was a measure of HIR Ab-LL-GLB1 fusion protein in the medium is >10 mg/L at a cell density of 10⁶/mLClones selected for dilutional cloning (DC) were removed from the orbital shaker in the incubator and transferred to the sterile hood. The cells were diluted to 500 mL in F-12K medium with 5% dialyzed fetal bovine serum (d-FBS) and Penicillin/Streptomycin, and the final dilution is 8 cells per mL, so that 4,000 wells in 40×96-well plates can be plated at a cell density of 1 cell per well (CPW). Once the cell suspension was prepared, within the sterile hood, a 125 uL aliquot was dispensed into each well of a 96-well plate using an 8-channel pipettor or a precision pipettor system. The plates were returned to the incubator at 37 C and 8% CO2. The cells diluted to 1 cell/well cannot survive without serum. On day 6 or 7, DC plates were removed from the incubator and transferred to the sterile hood where 125 μl of F-12K medium with 5% dialyzed fetal bovine serum (d-FBS) was added to each well. This selection media now contained 5% d-FBS, 30 nM MTX and 0.25 mg/mL Geneticin. On day 21 after the initial 1 CPW plating, aliquots from each of the 4,000 wells were removed for human IgG ELISA, using robotics equipment. DC plates were removed from the incubator and transferred to the sterile hood, where 100 μl of media was removed per well of the 96-well plate and transferred into a new, sterile sample 96-well plate using an 8-channel pipettor or the precision pipettor system. On day 20 after the initial 1 CPW plating, 40×96-well Immunoassay plates were plated with 100 uL of 1 μg/mL solution of Primary antibody, a mouse anti-human IgG in 0.1M NaHCO3. Plates are incubated overnight in the 4 C refrigerator. The following day, the ELISA plates were washed with 1×TBST 5 times, and 100 uL of 1 ug/mL solution of secondary antibody and blocking buffer were added. Plates are washed with 1×TBST 5 times. 100 uL of 1 mg/mL of 4-nitrophenyl phosphate di(2-amino-2-ethyl-1,3-propanediol) salt in 0.1M glycine buffer are added to the 96-well immunoassay plates. Plates were read on a microplate reader. The assay produced IgG output data for 4,000 wells/experiment. The highest producing 24-48 wells were selected for further propagation. The highest producing 24-well plates from the 1 CPW DC were transferred to the sterile hood and gradually subcloned through 6-well dishes, T75 flasks, and 125 mL square plastic bottles on an orbital shaker. During this process the serum was reduced to zero, at the final stage of centrifugation of the cells and resuspension in serum free medium (SFM). The above procedures were repeated with a second round of dilutional cloning, at 0.5-1 cells/well (CPW). At this stage, approximately 40% of the wells showed any cell growth, and all wells showing growth also secreted human IgG. These results confirmed that on average only 1 cell is plated per well with these procedures, and that the CHO cell line originates from a single cell. The dilutional cloning (DC) procedure was repeated as described above for a second round of DC. Cell lines generated from this second round DC were used for the preparation of the accession cell bank, to be later used in production of a Master Cell Bank. The CHO line producing the HIR Ab-GLB1 fusion protein was generated from CHO cells following 2 rounds of DC as described above. The accession cell line was propagated in a 2 L shake flask in SFM on an orbital shaker and the CHO-derived fusion protein was purified by protein A affinity chromatography.

### Example 6. Analysis of HIR Binding and GLB1 Activity of the Bi-Functional IgG-GLB1 Fusion Protein

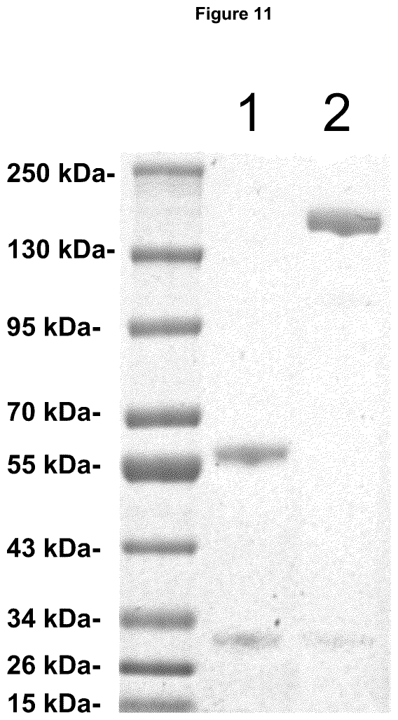

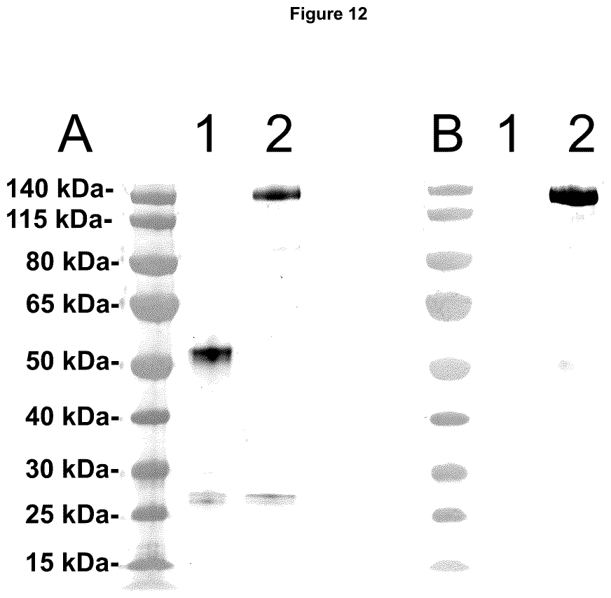

The HIR Ab-GLB1 fusion protein, following purification with protein A affinity chromatography, was assessed for purity by reducing sodium dodecysulfate polyacrylamide gel electrophoresis (SDS-PAGE) as shown in FIG. 11. Only the HC and LC proteins are detected for either the HIR Ab alone or the HIR Ab-GLB1 fusion protein. For the HIR Ab alone the higher molecular weight (MW) band is the HC and the lower MW band is the LC. For the HIR Ab-GLB1 fusion protein, the higher MW band is the HC-GLB1 fusion protein, and the lower MW band is the LC. The molecular weight (MW) of the HIR Ab-GLB1 heavy and light chains are estimated by linear regression based on the migration of the MW standards. The size of the HIR Ab-GLB1 fusion heavy chain, 140 kDa, is larger than the size of the heavy chain of the HIR Ab alone, 56 kDa, owing to the fusion of the GLB1 to the HIR Ab heavy chain. The size of the light chain, 27 kDa, is identical for both the HIR Ab-GLB1 fusion protein and the HIR Ab alone, as both proteins use the same light chain. The estimated MW of the hetero-tetrameric HIR Ab-GLB1 fusion protein shown in FIG. 2 is 334 kDa, based on migration in the SDS-PAGE of the Western blot. The identity of the fusion protein was verified by Western blotting using primary antibodies against either human IgG (FIG. 12A) or human GLB1 (FIG. 12B). The anti-human IgG antibody reacts with the HC and LC of both proteins, whereas the anti-GLB1 antibody only reacts with the HC of the fusion protein (FIG. 12).

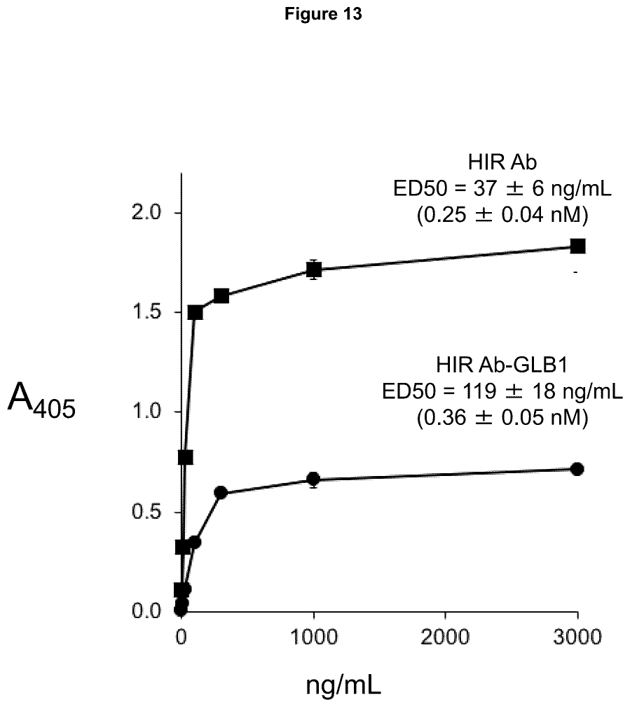

The potency of the CHO-derived fusion protein was assessed with the HIR ECD binding ELISA. The affinity of the fusion protein for the HIR extracellular domain (ECD) was determined with an ELISA. Recombinant HIR ECD was plated on Nunc-Maxisorb 96 well dishes and the binding of the HIR Ab, or the HIR Ab-GLB1 fusion protein, to the HIR ECD was detected with a secondary antibody, followed by binding with an alkaline phosphatase detector reagent. The concentration of either HIR Ab or HIR Ab-GLB1 fusion protein that gave 50% maximal binding, ED50, was determined by non-linear regression analysis. The ED50 of HIR Ab binding to the HIR is 37±6 ng/mL and the ED50 of Ab-GLB1 fusion protein binding to the HIR is 119±18 ng/mL (FIG. 13). The MW of the HIR Ab is 150 kDa, and the MW of the HIR Ab-fusion protein is 334 kDa. Therefore, after normalization for MW differences, there was comparable binding of either the chimeric HIR Ab or the HIR Ab-GLB1 fusion protein for the HIR ECD with ED50 of 0.25±0.04 nM and 0.36±0.05 nM, respectively (FIG. 13). These findings show that the affinity of the HIR Ab fusion protein binding to the HIR is retained, despite fusion of the GLB1 molecule to the carboxyl termini of both heavy chains of the IgG (FIG. 2).

The GLB1 enzyme activity was determined with a fluorometric assay using as substrate 4-methylumbelliferyl β-D-galactopyranoside (MUGP). This substrate is commercially available, and the structure of the substrate is outlined in FIG. 14A. This substrate is hydrolyzed by GLB1 to 4-methylumbelliferone (4MU), and 4MU formation in the assay is determined fluorometrically. The assay was performed by incubation of the HIR Ab-SL-GLB1 or the HIR Ab-LL-GLB1 fusion protein (25 to 250 ng/tube) and the MUGP (0.6 mM) in citrate buffer/pH=5.5 for 37 C for 20 minutes. The reaction was stopped by the addition of 0.5 M glycine/pH=10. Fluorescence was measured with a fluorometer with a 365 nm excitation filter and a 450 nm emission filter. A standard curve was generated with 10 to 3000 pmol/tube of the 4-methylumbelliferone (4MU) product, which allowed for conversion of fluorescent units to nmol/hr (FIG. 14B). The enzyme activity was measured as units/mg protein of the HIR Ab-SL-GLB1 or the HIR Ab-LL-GLB1 fusion protein, where 1 unit=1 nmol of 4MU product formed per hour of incubation. The assay was linear with respect to mass of fusion protein for either the HIR Ab-SL-GLB1 fusion protein or the HIR Ab-LL-GLB1 fusion protein (FIG. 14B). The GLB1 enzyme activity of the HIR Ab-LL-GLB1 fusion protein was 171±23 units/ug protein, or 171,000±23,000 units/mg protein. The GLB1 specific activity of the short linker fusion protein, HIR Ab-SL-GLB1, was reduced, 49±12 units/ug protein, or 49,000±12,000 units/mg protein. The specific activity of recombinant human GLB1 derived from CHO cells (R&D Systems) was also measured and was 183±56 units/ug protein, or 183,000±56,000 units/mg protein. The GLB1 enzyme activity of human recombinant GLB1 is comparable to the GLB1 enzyme activity reported for human GLB1 purified from human placenta, 203,000 units/mg protein (Hubbes, M., D'Agrosa, R. M., Callahan, J. W., Human placental beta-galactosidase. Characterization of the dimer and complex forms of the enzyme. Biochem J., 285: 827-831, 1992.), and comparable to the activity of feline recombinant GLB1 purified from CHO cells, 240,000 units/mg protein (Samoylova, T. I., et al, Generation and characterization of recombinant feline beta-galactosidase for preclinical enzyme replacement therapy studies in GM1 gangliosidosis, Metab. Brain Dis., 23: 161-173, 2008). The MW of the HIR Ab-LL-GLB1 fusion protein is 334 kDa, as discussed above, whereas the MW of recombinant GLB1 is 88 kDa [Zhang, S., et al, Kinetic mechanism and characterization of human beta-galactosidase precursor secreted by permanently transfected Chinese hamster ovary cells, Biochem. J., 304, 281-288, 1994.1. Since there are 2 GLB1 molecules per fusion protein tetramer (FIG. 2), the effective MW of the IgG-GLB1 is half of 344 kDa or 177 kDa, which is 2-fold greater than the MW of the recombinant GLB1. Therefore, on a molar basis, the GLB1 specific activity of the HIR Ab-LL-GLB1 fusion protein, 342,000 units/mg protein, is higher than the activity of the recombinant human GLB1. The GLB1 activity of the HIR Ab-SL-GLB1, on a molar basis, is equivalent to 98,000 units/mg protein.

### Example 7. Amino Acid Linker Joining the GLB1 and the Targeting Antibody

The mature human GLB1 is fused to the carboxyl terminus of the HC of the targeting antibody with a 31-amino acid linker (underlined in FIG. 10). This linker sequence corresponds to amino acids 462-492 of SEQ ID NO:11 (FIG. 10). The short 4-amino acid linker, which was associated with a decrease in enzyme activity, corresponds to amino acids 462-465 of SEQ ID NO:10 (FIG. 9). Any number of variations of linkers may be used as substitutions for the linker, both with respect to amino acid sequence and to amino acid length. Such linkers are well known in the art, as there are multiple publicly available programs for determining optimal amino acid linkers in the engineering of fusion proteins. A frequently used linker includes various combinations of Gly and Ser in repeating sequences, such as (Gly₄Ser)ₙ, or other variations. Such linkers may also be used when fusion of the GLB1 to the amino terminus of the LC of the targeting antibody, or when fusion of the GLB1 to the carboxy terminus of the HC of the targeting antibody, or when fusion of the GLB1 to the amino terminus of the HC of the targeting antibody, or when fusion of the GLB1 to either the amino terminus or the carboxy terminus of a single chain targeting antibody.

### Example 8. Receptor-Mediated Delivery of GLB1 to the Human Brain

GM1 gangliosidosis is caused by mutations in the GLB1 gene, which encodes the lysosomal enzyme galactosidase beta-1 (GLB1), which cleaves the terminal beta-galactose from GM1 ganglioside glycoconjugates. GLB1 deficiency in the brain, and in visceral organs, leads to the accumulation of glycolipids such as GM1 gangliosides. Such deposits in the brain causes a serious neurodegenerative inherited disease characterized by progressive cognitive decline, blindness, and seizures. Infantile GM1 leads to death in early childhood. Many such lysosomal storage diseases are treated with Enzyme Replacement Therapy (ERT) following expression of the recombinant enzyme. The sequence of the human GLB1 enzyme has been known for 30 years [Oshima et al (1988), “Cloning, sequencing, and expression of cDNA for human beta-galactosidase,” Biochem Biophys Res Comm 157: 238-2441. No ERT is currently FDA approved for treatment of the brain in GM1, because the GLB1 enzyme, like other lysosomal enzymes, does not cross the BBB. In an attempt to bypass the BBB, recombinant enzyme is given by intra-thecal (IT) delivery via direct injection into the cerebrospinal fluid (CSF) compartment of brain. Typically, the enzyme is injected into the lumbar or ventricular CSF space. The IT delivery route is not expected to be effective, because the enzyme is rapidly exported to the blood pool following injection into CSF. It is well known that the entire CSF volume turns over 4-5 times per day in humans, with export of the fluid, derived from the choroid plexus, back to the blood compartment. Drug injected into the CSF is equivalent to a slow intravenous injection, and drug injected into CSF only distributes to the ependymal surface of the brain, and not into the deep parenchymal tissue of brain where the enzyme is needed to correct the lipid storage disorder.

As disclosed herein, the preferred approach to the delivery of GLB1 to the brain of GM1 patients is the transvascular route of delivery following an intravenous (IV) infusion of a form of GLB1 that is re-engineered to cross the BBB via receptor-mediated transport (RMT). The HIR Ab-GLB1 fusion protein retains high affinity binding to the human insulin receptor, which enables the GLB1 to penetrate the BBB and enter brain from blood via RMT on the endogenous BBB insulin receptor. The HIR Ab-lysosomal enzyme fusion proteins is taken up by brain in the adult primate to produce a brain concentration of 1% of injected dose (ID) per brain, as well as even higher levels of uptake in visceral organs such as liver, spleen, and kidney [Boado et al (2013) Blood-brain barrier molecular Trojan horse enables brain imaging of radioiodinated recombinant protein in the Rhesus monkey. Bioconj. Chem., 24:1741-17491. If the therapeutic dose of the HIR Ab-GLB1 fusion protein is 3 mg/kg, and the body weight is 50 kg, then the infusion dose (ID) of the fusion protein is 150 mg. Given a brain uptake of the fusion protein of 1% of the ID, then the brain concentration of the fusion protein is 1500 ug/brain. This is equal to 1.5 ug/gram brain in the human, since the human brain weighs about 1,000 grams. The brain concentration of the HIR Ab-GLB1 fusion protein of 1.5 ug/gram is equivalent to 256 units/gram, since the GLB1 enzyme specific activity of the HIR Ab-GLB1 fusion protein is 171 units/ug protein, as described above. The endogenous GLB1 enzyme activity in the brain is 57±11 units/mg protein [Hahn, C. N., et al, Generalized CNS disease and massive GM1-ganglioside accumulation in mice defective in lysosomal acid beta-galactosidase, Human Molec Genet 6, 205-211, 1997)]. There are about 100 mg protein per gram brain, so the endogenous GLB1 enzyme activity in normal brain is 5700 units/gram. Therefore, the IV dose of 3 mg/kg of the HIR Ab-GLB1 fusion protein is expected to replace 256 units/g, or 4.5% of endogenous activity. This level of GLB1 enzyme activity in the brain is expected to be therapeutic, because it is recognized that restoration of as little as 1-2% of endogenous lysosomal enzyme activity is therapeutic in lysosomal storage disease (J. Muenzer and A. Fisher, Advances in the treatment of mucopolysaccharidosis type I, N. Engl J Med, 350: 1932-1934, 2004).These considerations show that a clinically significant GLB1 enzyme replacement of the human brain, and visceral organs, is possible following the intravenous infusion of the HIR Ab-GLB1 fusion protein at a systemic dose of approximately 3 mg/kg.

What is claimed:

1. A method for treating galactosidase beta-1 (GLB1) deficiency in a central nervous system of a subject in need thereof, comprising systemically administering to the subject a therapeutically effective dose of a fusion antibody having GLB1 activity, wherein the fusion antibody comprises:
(a) an amino acid sequence of galactosidase beta-1 (GLB1) set forth as SEQ ID NO:9,
(b) an immunoglobulin comprising a heavy chain and a light chain, wherein the immunoglobulin is capable of crossing the blood brain barrier (BBB) by binding to an endogenous BBB receptor-mediated transport system, and
(c) a linker between the amino acid sequence of the GLB1 and a carboxy terminus of the amino acid sequence of the immunoglobulin heavy chain,
wherein the GLB1 and the immunoglobulin each retains at least 20% of its activity compared to its activity as a separate entity, and wherein the amino acid sequence of the GLB1 is covalently linked to the carboxy terminus of the amino acid sequence of the immunoglobulin heavy chain.

2. The method of claim 1, wherein the linker is 80%, 85%, 90%, 95%, 96%, 97%, 98%, 99% or 100% identical to amino acids 462-492 of SEQ ID NO:11.

3. The method of claim 1, wherein the GLB1 activity of the fusion antibody is at least about 30,000 units/mg.

4. The method of claim 1, wherein the immunoglobulin heavy chain is an immunoglobulin heavy chain of IgG.

5. The method of claim 1, wherein the immunoglobulin heavy chain is an immunoglobulin heavy chain of IgG1 class.

6. The method of claim 1, wherein the immunoglobulin light chain is an immunoglobulin light chain of kappa or lambda class.

7. The method of claim 1, wherein the fusion antibody crosses the BBB via an endogenous BBB receptor selected from the group consisting of an insulin receptor, a transferrin receptor, a leptin receptor, a lipoprotein receptor, and an IGF receptor.

8. The method of claim 1, wherein the fusion antibody crosses the BBB by binding to an insulin receptor.

9. The method of claim 1, wherein the therapeutically effective dose of the fusion antibody comprises at least 0.5 mg/kg of body weight.
